SEQUENCE LISTING (3 sequences)

SEQ ID NO: 1 - DNA, 4059 bp, Artificial Sequence
  Description of Artificial Sequenceplasmid pBAT18
aatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgggtactggccacagagcttggcccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctgactctagcctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctctcgaggtcgacggtatcgataagcttgatatcgaattcctgcagcccgggggatccactagttctagagcggccgccaccgcggtggagctccacaactagaatgcagtgaaaaaaatgctttatttgtgaaatttgtgatgctattgctttatttgtaaccattataagctgcaataaacaagttaacaacaattgcattcattttatgtttcaggttcagggggaggtgtgggaggttttttaaagccacagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 2 - DNA, 4356 bp, Artificial Sequence
  Description of Artificial Sequenceplasmid pMB4
accgggccccccctcgaggtcgacggtatcgataagcttgatatcgaattcctgcagcccgggggatccactagttctagagcggccgccctagctctgaagttggtggtgaggccctgggcaggttggtatcaaggttacaagacaggtttaaggagaccaatagaaactgggcatgtggagacagagaagactcttgggtttctgataggcactgactctctctgcctattggtctattttcccacccttaggctgctggtctgagcctaggagatctgcgatctgcatctcaattagtcagcaaccatagtcccgcccctaactccgcccatcccgcccctaactccgcccagttccgcccattctccgccccatcgctgactaattttttttatttatgcagaggccgaggccgcctcggcctctgagctattccagaagtagtgaggaggcttttttggaggcctaggcttttgcaaaaagcttcgaatcgcgaattcgcccaccatgctgctgctgctgctgctgctgggcctgaggctacagctctccctgggcatcatcccagttgaggaggagaacccggacttctggaaccgcgaggcagccgaggccctgggtgccgccaagaagctgcagcctgcacagacagccgccaagaacctcatcatcttcctgggcgatgggatgggggtgtctacggtgacagctgccaggatcctaaaagggcagaagaaggacaaactggggcctgagatacccctggccatggaccgcttcccatatgtggctctgtccaagacatacaatgtagacaaacatgtgccagacagtggagccacagccacggcctacctgtgcggggtcaagggcaacttccagaccattggcttgagtgcagccgcccgctttaaccagtgcaacacgacacgcggcaacgaggtcatctccgtgatgaatcgggccaagaaagcagggaagtcagtgggagtggtaaccaccacacgagtgcagcacgcctcgccagccggcacctacgcccacacggtgaaccgcaactggtactcggacgccgacgtgcctgcctcggcccgccaggaggggtgccaggacatcgctacgcagctcatctccaacatggacattgacgtgatcctaggtggaggccgaaagtacatgtttcgcatgggaaccccagaccctgagtacccagatgactacagccaaggtgggaccaggctggacgggaagaatctggtgcaggaatggctggcgaagcgccagggtgcccggtatgtgtggaaccgcactgagctcatgcaggcttccctggacccgtctgtgacccatctcatgggtctctttgagcctggagacatgaaatacgagatccaccgagactccacactggacccctccctgatggagatgacagaggctgccctgcgcctgctgagcaggaacccccgcggcttcttcctcttcgtggagggtggtcgcatcgaccatggtcatcatgaaagcagggcttaccgggcactgactgagacgatcatgttcgacgacgccattgagagggcgggccagctcaccagcgaggaggacacgctgagcctcgtcactgccgaccactcccacgtcttctccttcggaggctaccccctgcgagggagctccatcttcgggctggcccctggcaaggcccgggacaggaaggcctacacggtcctcctatacggaaacggtccaggctatgtgctcaaggacggcgcccggccggatgttaccgagagcgagagcgggagccccgagtatcggcagcagtcagcagtgcccctggacgaagagacccacgcaggcgaggacgtggcggtgttcgcgcgcggcccgcaggcgcacctggttcacggcgtgcaggagcagaccttcatagcgcacgtcatggccttcgccgcctgcctggagccctacaccgcctgcgacctggcgccccccgccggcaccaccgacgccgcgcacccgggttactctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaattgcattcattttatgtttcaggttcagggggaggtgtgggaggttttttaaagccacagctccagcttttgttccctttagtgagggttaatttcgagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatatt

SEQ ID NO: 3 - protein, 9 aa, Artificial Sequence
  Description of Artificial SequenceHIV gp120 V3 loop peptide immunodominant epitope
GPGRAFYTT

(19) United States
(12) Patent Application Publication
(10) Pub. No.: US 2004/0115254 A1
(43) Pub. Date: Jun. 17, 2004

(54) Microcapsules and methods of use

(75) Inventors: Edmund J. Niedzinski, Vacaville, CA (US); Yen-Ju Chen, Alameda, CA (US); Yadong Liu, Fremont, CA (US); Eric Sheu, Lafayette, CA (US); Sean Tucker, San Francisco, CA (US)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: Genteric, Inc., Alameda, CA (US)

(21) Appl. No.: 10/655,702

(22) Filed: Sep. 5, 2003

Related U.S. Application Data
(60) Provisional application No. 60/408,646, filed on Sep. 6, 2002.
(60) Provisional application No. 60/424,882, filed on Nov. 8, 2002.
(60) Provisional application No. 60/458,661, filed on Mar. 28, 2003.

(51) Int. Cl. A61K 48/00; A61K 9/127

(52) U.S. Cl. 424/450; 514/44

(57) ABSTRACT

The present invention provides compositions and methods for making water-in-oil-in-water (w/o/w) microparticles. The microparticle comprises an active agent encapsulated in an aqueous interior, an amphiphilic binding molecule, and an encapsulation material. In certain preferred aspects, the amphiphilic binding molecule is a cationic lipid.

### CROSS-REFERENCES TO RELATED APPLICATIONS

This application claims priority to U.S. Provisional Patent Application No. 60/408,646, filed Sep. 6, 2002; 60/424,882, filed Nov. 8, 2002; and 60/458,661, filed Mar. 28, 2003, each of which is herein incorporated by reference in its entirety for all purposes.

### BACKGROUND OF THE INVENTION

Many systems for administering active substances into cells are already known, such as liposomes, nanoparticles, polymer particles, immuno- and ligand-complexes and cyclodextrins (see, Drug Transport in antimicrobial and anticancer chemotherapy. G. Papadakou Ed., CRC Press, 1995). However, none of these systems has proved to be truly satisfactory for the in vivo transport of nucleic acids such as, for example, deoxyribonucleic acid (DNA).

Satisfactory in vivo transport of nucleic acids into cells is necessary for example, in gene therapy. Gene transfer is the transfection of a nucleic acid-based product, such as a gene, into the cells of an organism. The gene is expressed in the cells after it has been introduced into the organism. Several methods of cell transfection exist at present. These methods include for example, use of calcium phosphate, microinjection, protoplasmic fusion; electroporation and injection of free DNA; viral infection; and synthetic vectors.

Gene delivery systems play an important role in human gene therapy. The foreign genes are required to be delivered into the target cells, and enter the nucleus for transcription and expression. Viral vector gene delivery systems have shown therapeutic level of gene expression and efficacy in animals and human clinical trials. Several kinds of viruses, including retrovirus, adenovirus, adeno-associated virus (AAV), and herpes simplex virus (HSV), have been manipulated for use in gene transfer and gene therapy applications. As different viral vector systems have their own unique advantages and disadvantages, they each have applications for which they are best suited. However, recent experiences with viral transfer of genes have shown the possible deleterious effects of viral gene delivery including inflammation of the meninges and potentially fatal reactions by the patient's immune system.

The processes to prepare viral vector gene delivery systems are complicated. Therefore, non-viral gene delivery systems have been extremely attractive and extensively investigated in the last 15 years. A number of lipid, peptide and polymer-based vectors have been designed. These delivery vectors show good transfection efficiency in cell cultures and the preparation methods are much easier than the viral delivery vectors. Cationic lipids show very good gene transfection in the lung. Some small molecules show enhancement in gene transfection in muscle. However, in vivo gene transfer is complicated by biological fluid interactions, immune clearance, toxicity and biodistribution, depending on the route of administration. Most of these non-viral gene carriers show poor in vivo gene expression, high toxicity and poor storage stability. In most cases, these vectors form DNA complex particles with negatively charged surface and usually show poor transfection activity, and the complexes with positive surface charge would bind with proteins in biological fluid to form big particles, or are even precipitated. This also decreases the biodistribution and transfection efficiency.

There is increasing interest in the use of synthetic vectors, such as lipid or polypeptide vectors. Synthetic vectors appear to be less toxic than the viral vectors. Among synthetic vectors, lipid vectors, such as liposomes, appear to have the advantage over polypeptide vectors of being potentially less immunogenic and, for the time being, more efficient. However, the use of conventional liposomes for DNA delivery is very limited because of the low encapsulation rate and their inability to compact large molecules, such as nucleic acids.

The formation of DNA complexes with cationic lipids has been studied by various laboratories (see, Felgner et al., PNAS 84, 7413-7417 (1987); Gao et al., Biochem. Biophys. Res. Commun. 179, 280-285, (1991); Behr, Bioconj. Chem. 5, 382-389 (1994)). These DNA-cationic lipid complexes have also been designated in the past using the term lipoplexes (see, P. Felgner et al., Hum. Genet. Ther., 8, 511-512, 1997). Cationic lipids enable the formation of relatively stable electrostatic complexes with DNA, which is a poylanionic substance.

Cationized polymers have also been investigated as vector complexes for transfecting DNA. For example, vectors called “neutraplexes” containing a cationic polysaccharide or oligosaccharide matrix have been described in U.S. application Ser. No. 09/126,402. Such vectors also contain an amphiphilic compound, such as a lipid.

U.S. Pat. No. 6,248,720 discloses microparticles that can be used to deliver oligonucleotides orally to the intestinal epithelium. The microparticles containing the oligonucleotides preferably are between 10 nanometers and five microns. The microparticles are prepared by phase inversion nanoencapsulation, and are thus limited in the amount of active agent that can be encapsulated.

In view of the above, there is a need for an improved vehicle for administering an active agent, such as a nucleic acid into a cell. There is also a need for improved methods for inducing tissue specific expression of the nucleic acid in a target cell. The present invention fulfills this and other needs.

### SUMMARY OF THE INVENTION

The present invention provides compositions and methods to formulate an active agent such as nucleic acid. In certain embodiments, the present invention provides multiple emulsion methods such as a water-in-oil-in-water (w/o/w) emulsion, to encapsulate nucleic acid for delivery into cells. The compositions and methods provide high encapsulation efficiency and controlled particle size. By using an amphiphilic binding molecule (ABM), it is possible, for example, to confine a hydrophilic active agent such as DNA, at the inner aqueous phase and to condense the active agent in a controllable manner.

As such, the present invention provides a particle comprising: an active agent optionally in an aqueous interior; an amphiphilic binding molecule; and an encapsulation material, wherein the amphiphilic binding molecule comprises a first functionality and a second functionality, wherein the first functionality has an affinity for the active agent and the second functionality is soluble in the same solvent as the encapsulation material.

In certain preferred aspects, the amphiphilic binding molecule is a cationic lipid. Suitable cationic lipids include, but are not limited to, N,N-dioleyl-N,N-dimethylammonium chloride (“DODAC”), N-(2,3-dioleyloxy)propyl)-N,N,N-trimethylammonium chloride (“DOTMA”), N,N-distearyl-N,N-dimethylammonium bromide (“DDAB”), N-(2,3-dioleoyloxy)propyl)-N,N,N-trimethylammonium chloride (“DOTAP”), 1,2-dimyristoyl-sn-glycero-3-trimethylammonium-propane (“DMTAP”), 1,2-dipalmitoyl-sn-glycero-3-trimethylammonium-propane (“DPTAP”), and 1,2-distearoyl-sn-glycero-3-trimethylammonium-propane (“DSTAP”), 3-(N-(N′,N′-dimethylaminoethane)-carbamoyl)cholesterol (“DC-Chol”), N-(1,2-dimyristyloxyprop-3-yl)-N,N-dimethyl-N-hydroxyethyl ammonium bromide (“DMRIE”), 1,2-dilauroyl-P-O-ethylphosphatidylcholine (“E-DLPC”), 1,2-dimyristoyl-P-O-ethylphosphatidylcholine (“E-DMPC”), 1,2-dipalmitoyl-P-O-ethylphosphatidylcholine (“E-DPPC”), and mixtures thereof.

In certain other preferred aspects, the encapsulation material is a hydrophobic polymer. Suitable hydrophobic polymers include, but are not limited to, poly(lactid-co-glycolide), poly(lactic acid), poly(caprolactone), poly(glycolic-acid), poly(anhydrides), poly(orthoesters), poly(hydroxybutyric acid), poly(alkylcyanoacrylate), poly(lactides), poly(glycolides), poly(lactic acid-co-glycolic acid), polycarbonates, polyesteramides, poly(amino acids), polycyanoacrylates, poly(p-dioxanone), poly(alkylene oxalate), biodegradable polyurethanes, blends, and mixtures thereof.

In some embodiments, the particle further comprises a stabilizing agent. Suitable stabilizing agents include, but are not limited to, polyvinyl alcohol (PVA), methylcellulose, hydroxyethyl cellulose, hydroxypropylmethylcellulose, gelatin, a carbomer, and a poloxamer. In some embodiments, the particle further comprises an enteric coating.

In another embodiment, the present invention provides a process for preparing a particle, comprising: admixing a first aqueous solution having an active agent with an organic solvent having an encapsulation material to form an emulsion; admixing an amphiphilic binding molecule with the emulsion to form an amphiplex; and admixing the amphiplex with a second aqueous solution having a stabilizing agent to form a particle, wherein the amphiphilic binding molecule comprises a first functionality and a second functionality, wherein the first functionality has an affinity for the active agent and the second functionality is soluble in the same solvent as the encapsulation material. In certain embodiments, the present invention provides a particle made by such method. In a preferred embodiment, the process for preparing a particle further comprises lyophilizing the particle to form a delivery particle.

In a preferred aspect, increasing the amphiphilic binding molecule concentration (e.g., cationic lipid) decreases the diameter of the particle. In another preferred aspect, increasing the amphiphilic binding molecule concentration (e.g., cationic lipid) increases the encapsulation efficiency of the active agent. In yet another preferred aspect, the use of amphiphilic binding molecules (e.g., cationic lipids) with longer hydrophobic domains decreases the diameter of the particle. In still yet another preferred aspect, the use of amphiphilic binding molecules (e.g., cationic lipids) with longer hydrophobic domains increases the encapsulation efficiency of the active agent.

In certain aspects, the present methods are based upon water-in-oil-in-water (w/o/w) emulsion techniques. In certain aspects, an active agent, such as an oligonucleotide in an aqueous solution, is added to an organic solution containing an encapsulation material such as a polymer (e.g., hydrophobic or hydrophilic polymer). This solution is then emulsified and an amphiphilic binding agent is then added. This resulting mixture is emulsified and thereafter added to an aqueous solution that optionally contains a stabilizing agent, such as PVA. In one aspect, the solution is stirred until the organic layer evaporates, allowing the polymer to precipitate onto a surface, such as a droplet containing an active agent. In certain preferred aspects, the active agent is a nucleic acid. Suitable nucleic acids include, but are not limited to, DNA, RNA, DNA/RNA hybrids, an antisense oligonucleotide, siRNA (small inhibitory RNA), a chimeric DNA-RNA polymer, a ribozyme, and plasmid DNA. In some embodiments, the nucleic acid comprises a sequence encoding a therapeutic protein. In certain embodiments, the therapeutic protein is interferon α, interferon β, interferon γ, or insulin. Preferably, the therapeutic protein is interferon β. In some embodiments, the nucleic acid is operably linked to a tissue specific expression control sequence. In certain aspects, the expression control sequence is tissue specific. Suitable tissues include, but are not limited to, intestinal epithelium, liver, lung, pancreas, breast, brain, and muscle. Preferably, the tissue is intestinal epithelium or liver.

A further embodiment of the present invention provides a delivery particle comprising: an inner core having an active agent; an amphiphilic binding molecule; and a polymeric outer layer, wherein the amphiphilic binding molecule is situated between the inner core and the outer layer. In certain aspects, the inner core comprises an active agent in a disperse phase. In other aspects, the inner core comprises a disperse phase, an active agent, or a mixture of an outer layer and an active agent. In yet another aspect, the polymeric outer layer is an organic phase.

Another embodiment of the present invention provides a method for delivering an active agent to a subject by administering a particle as described herein to the subject. In certain aspects, the administration is oral. In certain aspects, the active agent is a nucleic acid. In certain preferred aspects, the nucleic acid encodes a therapeutic protein. Suitable therapeutic proteins include, but are not limited to, interferon α, interferon β, interferon γ, and insulin. In a further aspect, the nucleic acid is operably linked to an expression control sequence. In one aspect, the therapeutic protein is not expressed in an intestinal epithelial cell. In a preferred aspect, the therapeutic protein is expressed in an intestinal epithelial cell. In certain aspects, the expression control sequence is tissue specific. In a preferred aspect, the tissue is intestinal epithelium.

Yet another embodiment of the invention provides a method for treating a subject with a disease by administering a particle as described herein to the subject. In certain aspects, the administration is oral. In certain aspects, the active agent is a nucleic acid. In certain preferred aspects, the nucleic acid encodes a therapeutic protein. In a further aspect, the nucleic acid is operably linked to an expression control sequence. In one aspect, the therapeutic protein is not expressed in an intestinal epithelial cell. In a preferred aspect, the therapeutic protein is expressed in an intestinal epithelial cell. In certain aspects, the expression control sequence is tissue specific. In a preferred aspect, the tissue is intestinal epithelium. Suitable diseases that can be treated with a particle of the present invention include, but are not limited to, autoimmune disorders, protein deficiency disorders, blood disorders, cardiovascular disorders, central nervous system disorders, gastrointestinal disorders, metabolic disorders, neoplastic diseases, pulmonary disorders, and bacterial and viral diseases.

An even further embodiment of the invention provides a method for inducing an immune response in a subject by administering a particle as described herein to the subject. In certain aspects, the administration is oral. In certain aspects, the active agent is a nucleic acid. In a further aspect, the nucleic acid is operably linked to an expression control sequence. In one aspect, the nucleic acid is not expressed in an intestinal epithelial cell, but in a cell residing within the intestine, either temporarily or permanently. Suitable examples include, but are not limited to, dendritic cells and lymphocytes. In other aspects, the nucleic acid is expressed in an intestinal epithelial cell. Suitable antigens encoded by the nucleic acid for inducing an immune response include, but are not limited to, a bacterial antigen, a viral antigen, a fungal antigen, and a parasitic antigen. In certain aspects, the expression control sequence is tissue specific. In a preferred aspect, the tissue is intestinal epithelium.

In certain other instances, the present invention provides for the use of a particle in the manufacture of medicament for the delivery of an active agent.

These and other embodiments and aspects will become more apparent when read with the accompanying drawings and the detailed description, which follow.

### DESCRIPTION OF THE DRAWINGS

FIG. 1 shows a schematic of a method according to one embodiment for the present invention.

FIG. 2 shows a schematic according to one embodiment for the present invention.

FIG. 3 shows one embodiment of a microparticle of the present invention.

FIG. 4 shows one embodiment of a microparticle of the present invention.

FIG. 5 shows the effect of lipid structure on the concentration of DNA within PLG microparticles.

FIG. 6 shows the effect of lipid structure on DNA encapsulation efficiency.

FIGS. 7A-B show the effect of lipid structure on particle size. Panel A shows the effect of E-DLPC, E-DMPC, and E-DPPC on particle size. Panel B shows the effect of DMTAP, DPTAP, DSTAP, and DOTAP on particle size.

FIG. 8 shows the effect of cationic lipid concentration on DNA encapsulation efficiency.

FIG. 9 shows the effect of cationic lipid concentration on particle size.

FIG. 10 illustrates an analysis of DNA integrity after extraction from microparticles.

FIG. 11 illustrates a particle size analysis of cationic lipid-microparticle formulation.

FIG. 12 shows the concentration of extracellular DNA following administration of the cationic lipid-microparticle formulation to CHO cells.

FIG. 13 shows an analysis of transfection efficiency in CHO cells at 24, 48, and 120 hours (h) after administration of the cationic lipid-microparticle formulation.

FIG. 14 shows the particle sizes and encapsulation efficiencies of cationic lipid-microparticle formulations containing other active ingredients other than DNA.

FIG. 15 illustrates an antibody response to human growth hormone (hGH) following delivery of DNA encoding hGH.

FIG. 16 illustrates a response to HIV gp120 following delivery of DNA encoding HIV gp120 and an antibody response to HIV gp120.

FIG. 17 illustrates a CTL response to HIV gp120.

FIG. 18 illustrates expression of IFNβ using the vector constructed as described in Example X below.

FIG. 19 is a graphic illustration of a pBAT18 vector.

FIG. 20 is a graphic illustration of a pMB4 vector.

### DETAILED DESCRIPTION OF THE INVENTION AND PREFERRED EMBODIMENTS

I. Definitions

The terms “nucleic acid” and “polynucleotide” are used interchangeably herein to refer to deoxyribonucleotides or ribonucleotides and polymers thereof in either single- or double-stranded form. The term encompasses nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Nucleotides may be referred to by their commonly accepted single-letter codes. These are A, adenine; C, cytosine; G, guanine; and T, thymine (DNA), or U, uracil (RNA).

The term “codon” refers to a sequence of nucleotide bases that specifies an amino acid or represents a signal to initiate or stop a function. Unless otherwise indicated, a particular nucleic acid sequence also encompasses conservatively modified variants thereof (e.g., degenerate codon substitutions) and complementary sequences, as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al, J. Biol. Chem. 260:2605 (1985); Rossolini et al., Mol. Cell. Probes 8:91 (1994)). The term nucleic acid is used interchangeably with gene, cDNA, mRNA, oligonucleotide, and polynucleotide.

DNA may be in the form of anti-sense, plasmid DNA, parts of a plasmid DNA, the product of a polymerase chain reaction (PCR), vectors (P1, PAC, BAC, YAC, artificial chromosomes), expression cassettes, chimeric sequences, chromosomal DNA, or derivatives of these groups. RNA may be in the form of oligonucleotide RNA, tRNA (transfer RNA), snRNA (small nuclear RNA), rRNA (ribosomal RNA), mRNA (messenger RNA), siRNA (small inhibitory RNA), anti-sense RNA, ribozymes, chimeric sequences, or derivatives of these groups.

“Antisense” is a polynucleotide that interferes with the function of DNA and/or RNA. This may result in suppression of expression. Natural nucleic acids have a phosphate backbone, artificial nucleic acids may contain other types of backbones and bases. These include PNAs (peptide nucleic acids), phosphothionates, and other variants of the phosphate backbone of native nucleic acids. In addition, DNA and RNA may be single, double, triple, or quadruple stranded.

The term “gene” refers to a nucleic acid (e.g., DNA) sequence that comprises coding sequences necessary for the production of a polypeptide or precursor (e.g., myosin heavy chain). The polypeptide can be encoded by a full length coding sequence or by any portion of the coding sequence so long as the desired activity or functional properties (e.g., enzymatic activity, ligand binding, signal transduction, and the like) of the full-length or fragment are retained. The term also encompasses the coding region of a structural gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ non-translated sequences. The sequences that are located 3′ or downstream of the coding region and which are present on the mRNA are referred to as 3′ nontranslated sequences. The term “gene” encompasses both cDNA and genomic forms of a gene. A genomic form or clone of a gene contains the coding region interrupted with noncoding sequences termed “introns” or “intervening regions” or “intervening sequences.” Introns are segments of a gene, which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Upregulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

A “therapeutic protein” or “therapeutic nucleic acid” is any protein or nucleic acid that provides a therapeutic, prophylactic effect, or both. A therapeutic protein may be naturally occurring or produced by recombinant means. A “therapeutically effective amount” of a nucleic acid or protein is an amount of nucleic acid or protein sufficient to provide a therapeutic or prophylactic effect in a subject. Such therapeutic or prophylactic effects may be local or systemic. Therapeutic and prophylactic effects include, for example, restoring or enhancing a normal metabolic response; or eliciting or modulating an immune response. Selby et al. (2000) J. Biotechnol. 83(1-2):147-52. Normal metabolic responses include secretion of insulin and glucagons in response to changing blood sugar levels. Immune responses include humoral immune responses and cell-mediated immune responses. (see, Fundamental Immunology (Paul ed., 4th ed. 1999).

The terms “polypeptide,” “peptide” and “protein” are used interchangeably herein to refer to a polymer of amino acid residues. The terms apply to naturally occurring amino acid polymers, as well as, amino acid polymers in which one or more amino acid residue is an artificial chemical mimetic of a corresponding naturally occurring amino acid.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified through post translational modification, e.g., hydroxyproline, γ-carboxyglutamate, and O-phosphoserine. “Amino acid analogs” refers to compounds that have the same fundamental chemical structure as a naturally occurring amino acid, i.e., an alpha carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. “Amino acid mimetics” refers to chemical compounds that have a structure that is different from the general chemical structure of an amino acid, but that functions in a manner similar to a naturally occurring amino acid. Amino acids may be referred to herein by either their commonly known three letter symbols or by the one-letter symbols recommended by the IUPAC-IUB Biochemical Nomenclature Commission.

“Conservatively modified variants” applies to both nucleic acid and amino acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

With respect to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologues, and alleles of the invention.

Each of the following eight groups contains amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W);

7) Serine (S), Threonine (T); and

8) Cysteine (C), Methionine (M)

(see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3rd ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 50 to 350 amino acids long. Typical domains are made up of sections of lesser organization such as stretches of β-sheet and α-helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

A “label” or “detectable label” is a composition detectable by spectroscopic, photochemical, biochemical, immunochemical, or chemical means. For example, useful labels include radioisotopes (e.g., ³H, ³⁵S, ³²P, ⁵¹ Cr, or ¹²⁵I), fluorescent dyes, electron-dense reagents, enzymes (e.g., alkaline phosphatase, horseradish peroxidase, or others commonly used in an ELISA), biotin, digoxigenin, or haptens and proteins for which antisera or monoclonal antibodies are available.

The term “recombinant” when used with reference, e.g., to a cell, or nucleic acid, protein, or vector, indicates that the cell, nucleic acid, protein or vector, has been modified by the introduction of a heterologous nucleic acid or protein or the alteration of a native nucleic acid or protein, or that the cell is derived from a cell so modified. Thus, for example, recombinant cells express genes that are not found within the native (non-recombinant) form of the cell or express native genes that are otherwise abnormally expressed, under expressed or not expressed at all.

The terms “promoter” and “expression control sequence” are used herein to refer to an array of nucleic acid control sequences that direct transcription of a nucleic acid. As used herein, a promoter includes necessary nucleic acid sequences near the start site of transcription, such as, in the case of a polymerase II type promoter, a TATA element. A promoter also optionally includes distal enhancer or repressor elements, which can be located as much as several thousand base pairs from the start site of transcription. A “constitutive” promoter is a promoter that is active under most environmental and developmental conditions. An “inducible” promoter is a promoter that is active under environmental or developmental regulation. The term “operably linked” refers to a functional linkage between a nucleic acid expression control sequence (such as a promoter, or array of transcription factor binding sites) and a second nucleic acid sequence, wherein the expression control sequence directs transcription of the nucleic acid corresponding to the second sequence.

The term “heterologous” when used with reference to portions of a nucleic acid indicates that the nucleic acid comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source. Similarly, a heterologous protein indicates that the protein comprises two or more subsequences that are not found in the same relationship to each other in nature (e.g., a fusion protein).

An “expression vector” or “expression cassette” is a nucleic acid construct, generated recombinantly or synthetically, with a series of specified nucleic acid elements that permit transcription of a particular nucleic acid in a host cell. The expression vector can be part of a plasmid, virus, or nucleic acid fragment. Typically, the expression vector includes a nucleic acid to be transcribed operably linked to a promoter.

As used herein, the term “aqueous phase” refers to a composition comprising in whole, or in part, water.

The term “lipid” refers to a group of organic compounds that are esters such as fatty acid esters, and are characterized by being insoluble in water but soluble in many organic solvents. They are usually divided in at least three classes: (1) “simple lipids” which include fats and oils as well as waxes; (2) “compound lipids” which include phospholipids and glycolipids; (3) “derived lipids” such as steroids.

The term “amphipathic lipid” refers, in part, to any suitable material wherein the hydrophobic portion of the lipid material orients into a hydrophobic phase, while a hydrophilic portion orients toward the aqueous phase. Amphipathic lipids are usually the major component of a lipid vesicle. Hydrophilic characteristics derive from the presence of polar or charged groups such as carbohydrates, phosphato, carboxylic, sulfato, amino, sulfhydryl, nitro, hydroxy and other like groups. Hydrophobicity can be conferred by the inclusion of apolar groups that include, but are not limited to, long chain saturated and unsaturated aliphatic hydrocarbon groups and such groups substituted by one or more aromatic, cycloaliphatic or heterocyclic group(s). Examples of amphipathic compounds include, but are not limited to, phospholipids, aminolipids and sphingolipids. Representative examples of phospholipids include, but are not limited to, phosphatidylcholine, phosphatidylethanolamine, phosphatidylserine, phosphatidylinositol, phosphatidic acid, palmitoyloleoyl phosphatidylcholine, lysophosphatidylcholine, lysophosphatidylethanolamine, dipalmitoylphosphatidylcholine, dioleoylphosphatidylcholine, distearoylphosphatidylcholine or dilinoleoylphosphatidylcholine. Other compounds lacking in phosphorus, such as sphingolipid, glycosphingolipid families, diacylglycerols and β-acyloxyacids, are also within the group designated as amphipathic lipids. Additionally, the amphipathic lipid described above can be mixed with other lipids including triglycerides and sterols.

The term “anionic lipid” refers to any lipid that is negatively charged at physiological pH. These lipids include, but are not limited to, phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, and other anionic modifying groups joined to neutral lipids.

The term “cationic lipid” refers to any of a number of lipid species, which carry a net positive charge at a selective pH, such as physiological pH. Such lipids include, but are not limited to, N,N-dioleyl-N,N-dimethylammonium chloride (“DODAC”), N-(2,3-dioleyloxy)propyl)-N,N,N-trimethylammonium chloride (“DOTMA”), N,N-distearyl-N,N-dimethylammonium bromide (“DDAB”), 1,2-dimyristoyl-sn-glycero-3-trimethylammonium-propane (“DMTAP”), 1,2-dipalmitoyl-sn-glycero-3-trimethylammonium-propane (“DPTAP”), and 1,2-distearoyl-sn-glycero-3-trimethylammonium-propane (“DSTAP”), 3-(N-(N′,N′-dimethylaminoethane)-carbamoyl)cholesterol (“DC-Chol”), N-(1,2-dimyristyloxyprop-3-yl)-N,N-dimethyl-N-hydroxyethyl ammonium bromide (“DMRIE”), 1,2-dilauroyl-P-O-ethylphosphatidylcholine (“E-DLPC”), 1,2-dimyristoyl-P-O-ethylphosphatidylcholine (“E-DMPC”), 1,2-dipalmitoyl-P-O-ethylphosphatidylcholine (“E-DPPC”), and N-(2,3-dioleoyloxy)propyl)-N,N,N-trimethylammonium chloride (“DOTAP”). Additionally, a number of commercial preparations of cationic lipids are available which can be used in the present invention. These include, for example, LIPOFECTIN® (commercially available cationic liposomes comprising DOTMA and 1,2-dioleoyl-sn-3-phosphoethanolamine (“DOPE”), from GIBCO/BRL, Grand Island, N.Y., USA); LIPOFECTAMINE® (commercially available cationic liposomes comprising N-(1-(2,3-dioleyloxy)propyl)-N-(2-(sperminecarboxamido)ethyl)-N,N-dimethylammonium trifluoroacetate (“DOSPA”) and (“DOPE”), from GIBCO/BRL); and TRANSFECTAM® (commercially available cationic lipids comprising dioctadecylamidoglycyl carboxyspermine (“DOGS”) in ethanol from Promega Corp., Madison, Wis., USA). The following lipids are cationic and have a positive charge at below physiological pH: DODAP, DODMA, DMDMA, and the like.

As used herein, the terms “microparticle,” “particle,” “delivery particle,” “delivery microparticle” and the like, refer to a composition that can be used to deliver an active agent, either in solution or as a solid, wherein the active agent is surrounded by an encapsulation material, preferably having an amphiphilic binding agent therebetween. The encapsulation material coats an interior comprising an active agent, such as a plasmid.

As used herein, “encapsulation” can refer to a formulation that provides a compound with full encapsulation, partial encapsulation, or combinations thereof

As used herein, the term “amphiplex” means an emulsion between an aqueous solution and an organic solvent, wherein the emulsion further comprises an amphiphilic binding molecule.

As used herein, the term “encapsulation material” or “coating” means a material that can be used to embed, in whole or in part, an active agent. Preferred encapsulation materials include, but are not limited to, hydrophobic polymers, hydrophilic polymers, lipids, natural or synthetic polymers and surfactants, and combinations thereof. Hydrophobic polymers are preferred encapsulation materials. Suitable hydrophobic polymers include, but are not limited to, poly(lactid-co-glycolide), poly(lactic acid), poly(caprolactone), poly(glycolic-acid), poly(anhydrides), poly(orthoesters), poly(hydroxybutyric acid), poly(alkylcyanoacrylate), poly(lactides), poly(glycolides), poly(lactic acid-co-glycolic acid), polycarbonates, polyesteramides, poly(amino acids), polycyanoacrylates, poly(p-dioxanone), poly(alkylene oxalate), biodegradable polyurethanes, blends, and mixtures thereof.

As used herein, the term “charge ratio” refers for example, to the moles of cationic lipid that is added to the formulation per mole of phosphate group in the DNA backbone.

As used herein, the term “inner core” refers to the center or middle region of a microparticle or particle, which may or may not comprise an aqueous interior, wherein the active agent predominately resides. In certain instances, the inner core is surrounded by an encapsulation material.

As used herein, the term “amphiphilic binding molecule is situated” means that the amphiphilic binding molecule (e.g., a cationic lipid) resides at the interface between a first phase and a second phase, for example, between an inner core and a polymeric outer layer, with the hydrophilic end complexed with DNA through, for example, a charge-charge interaction or a hydrophilic interaction, and the lipophilic end immersed and/or dissolved and/or embedded in an immiscible phase (e.g., an oil phase).

II. Methods of Microparticle Preparation

In one embodiment, the present invention provides a process for preparing a microparticle, the method comprising: admixing a first aqueous solution having an active agent with an organic solvent having an encapsulation material to form an emulsion; admixing an amphiphile binding molecule with the emulsion to form an amphiplex; and admixing the amphiplex with a second aqueous solution having a stabilizing agent to form a microparticle having an encapsulated active agent. As will be apparent to those of skill in the art, the order of mixing and adding the various components can be varied so that the optimum products can be formed.

In certain aspects, the present methods are based upon water-in-oil-in-water (w/o/w) emulsion techniques. In certain aspects, the oligonucleotide is added to an organic solution containing a polymer, such as a hydrophobic polymer. In certain aspects, this solution is then emulsified and an amphiphilic binding molecule (ABM) is then added. The resulting mixture is emulsified and then added to an aqueous solution optionally containing a stabilizing agent, such as polyvinyl alcohol (PVA). The solution is stirred until the organic layer evaporates, allowing the polymer to precipitate onto a surface, such as an aqueous layer containing an active agent.

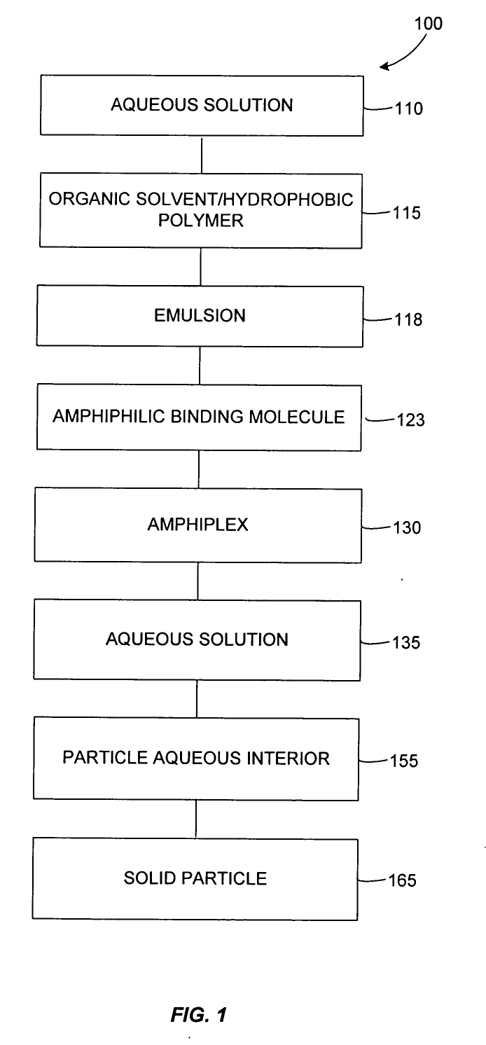

FIG. 1 is an example of a representative flow chart (100) of a method of the present invention. This flow chart is merely an illustration and should not limit the scope of the claims herein. One of ordinary skill in the art will recognize other variations, modifications, and alternatives.

As shown therein, a first aqueous solution (110) comprising an active agent (e.g., nucleic acid) is added to an organic solvent having an encapsulation material (115) to form an emulsion (118). Thereafter, an amphiphilic binding molecule (123) is mixed with the emulsion to form an amphiplex (130). The amphiplex is mixed with a second aqueous solution (135) optionally having a stabilizing agent to form a particle (155) having an encapsulated active agent and an aqueous interior. A solid delivery particle (165) is produced after lyophilization. As will be apparent to one of skill in the art, the exact order of steps can be changed to effectuate the resulting particles. For example, in one aspect, the ABM is added to the aqueous solution of (110), and is present in the emulsion of (118).

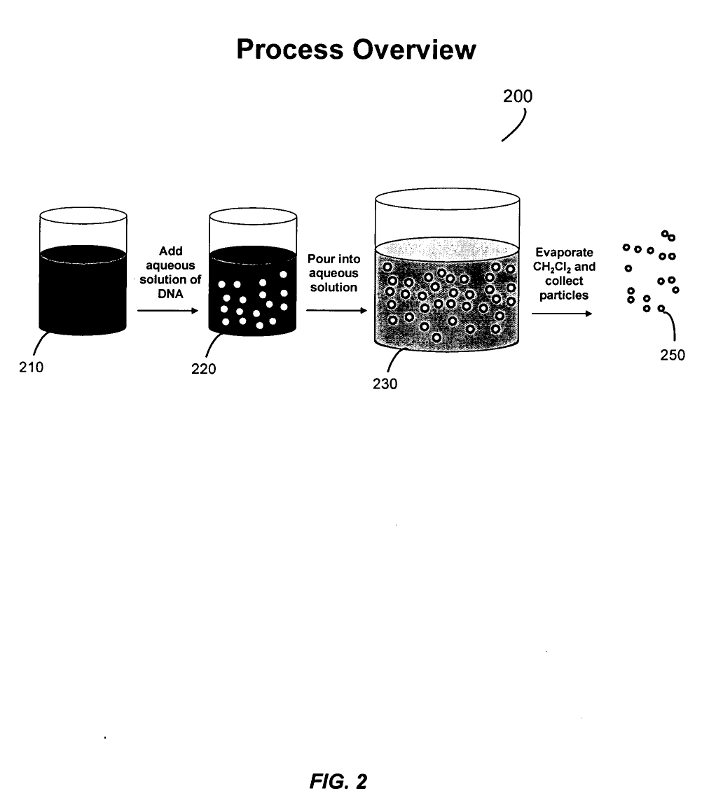

FIG. 2 shows an illustrative schematic (200) of a method of the present invention. As shown therein, in one embodiment, the process is a double emulsion process, wherein an encapsulating material is dissolved in an organic solvent such as PLG dissolved in methylene chloride (210). To this organic solution, an aqueous solution is added, such as an aqueous solution comprising an active agent (e.g., DNA) (220) to produce a water-in-oil (w/o) emulsion. The w/o emulsion is added to an aqueous solution to produce a water-in-oil-in water (w/o/w) emulsion (230). After evaporation of the organic solvent (e.g., methylene chloride), delivery microparticles are produced (250).

The methods of the present invention can be preferably used for making w/o/w emulsions. However, the methods are not so limited and can be used in w/o, o/w, o/w/o and combinations thereof. This flexibility leads to a wide range of applications and uses.

In one exemplary w₁/o/w₂ emulsion, the amphiphilic binding molecule (e.g., cationic lipid) is situated between the w₁/o phase. The ABM prevents or retards the active agent in w₁ from going into w₂ during the process of phase evaporation. At the end of the evaporation process, the “o” phase will disappear to form a solid polymer shell or protective coating that encapsulates or embeds the active agent in w₁ In one embodiment, the ABM is situated at the o/w₂ interface, which has the same effect on encapsulating the active agent.

The use of an ABM is also useful for super critical fluid (SCF) and spray drying processes. For example, in SCF processes there are two phases to start with, wherein the active agent is dissolved in the water phase, and super critical CO₂ acts as the oil phase in the outer phase containing an encapsulating polymer. The ABM resides in the interface. As it depressurizes, the CO₂ disappears leaving only the solid polymer sphere containing water with the active agent in it. The function of the ABM in this case is to maintain the integrity and/or structure of the disperse phase (e.g., water) through the depressurizing process.

As such, in yet another embodiment, the present invention provides a method for retaining a material in a first phase of a two phase system, comprising: providing an amphiphilic binding molecule comprising a first functionality and a second functionality, wherein the first functionality has an affinity for the material in the first phase and the second functionality is soluble in a second phase; and wherein the amphiphilic binding molecule is situated or traverses the first phase and the second phase. This allows the ABM to retain the material in the first phase. In certain preferred aspects, the first phase is a disperse phase. Preferably, the second phase is immiscible in the first phase. In another embodiment, the two phase system further comprises a third phase to generate a three phase system, such as a w₁/o/w₂ emulsion. In certain preferred aspects, the amphiphilic binding molecule is a cationic lipid. In certain other preferred aspects, the material is an active agent. Preferably, the active agent is nucleic acid.

III. Compositions

In other embodiments, the present invention provides a microparticle comprising an active agent optionally in an aqueous interior; an amphiphilic binding molecule (ABM); and an encapsulation material, wherein the amphiphilic binding molecule comprises a first functionality and a second functionality, wherein the first functionality has an affinity for the active agent and the second functionality is soluble in the same solvent as the encapsulating material.

In certain aspects, the present invention provides a water-in-oil-in-water (w/o/w) microparticle prepared by processes as described herein. In certain aspects, the microparticle comprises an active agent encapsulated in an aqueous interior; an ABM, and an encapsulation material such as a hydrophobic polymeric coating. In certain preferred aspects, the ABM is a molecule, for example, having dual functionalities or properties, such as opposite properties on each end of the molecule. In one aspect, the first functionality has an affinity for the active agent and the second functionality of the ABM is soluble in the same solvent as the encapsulating material. For example, one end of the molecule is for “holding” the active agent in the inner aqueous phase, while the other end has an affinity or is soluble in the middle oil phase, comprising the encapsulating material.

The first functionality of the ABM has an affinity for the active agent. For example, if the active agent is nucleic acid having a negative charge, the first functionality of the ABM can be a functional group carrying a positive charge, such as a cationic lipid or a conjugated cationic lipid (e.g., PEG-lipid). The second functionality of the ABM is soluble in the same solvent as the encapsulating material. For example, if the encapsulating material is a hydrophobic polymer soluble in for instance, a chlorinated hydrocarbon (e.g., methylene chloride), the second functionality is soluble in the chlorinated hydrocarbon as well. As used herein, the term “soluble” pertains to phases that mix to form a homogeneous mixture.

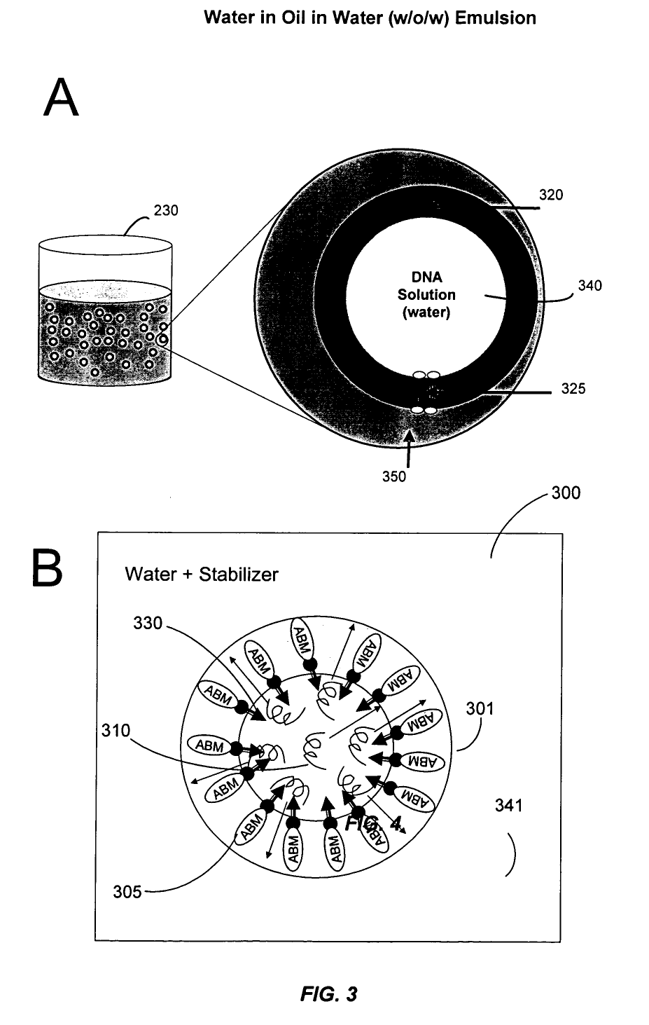

FIG. 3 is a diagram of a representative embodiment of a composition of the present invention. This diagram is merely an illustration and should not limit the scope of the claims herein. One of ordinary skill in the art will recognize other variations, modifications, and alternatives.

FIG. 3A is an expanded view of item (230) in FIG. 2. In the process described above, the w/o emulsion is added to an aqueous solution to produce a water-in-oil-in water (w/o/w) emulsion (230). In certain embodiments, during the w/o/w process, the active agent is contained within a “droplet” rather than for example, a particle. The droplet has two phases. The inner aqueous phase contains DNA in a “dissolved” state. The aqueous droplet is coated with an oil layer containing the encapsulation material. The ABM is situated in between with one end “interacting” with DNA through for example, a charge-charge interaction, while the other end (e.g., the hydrophobic portion) is embedded (or dissolved) in the oil phase layer wherein the encapsulation material is dissolved. This two-layer droplet is “dispersed” in the w₂ aqueous phase that preferably contains a stabilizer. In the expanded view of FIG. 3A, the w/o/w emulsion comprises a droplet having an amphiphilic binding molecule (325), which is situated between both the w₁/o phase and the o/w₂ interface. In this embodiment, the ABM (325) traverses the encapsulation material and solvent (320) with functionalities in both water phases (340) and (350).

As shown in FIG. 3B, an active agent (310) such as DNA, is dissolved in an aqueous interior phase. An amphiphilic binding molecule (305) such as a cationic lipid, surrounds the active agent (310) and holds the active agent in the aqueous phase using for example, a charge-charge interaction or a hydrophilic interaction (330). The other end of the ABM has an affinity for the middle oil phase wherein the encapsulation material is dissolved (301). In certain embodiments, the ABM is at the interface, or situated between, the active agent (310) and the encapsulation material (301). The encapsulation material can be a hydrophobic polymer coating. Preferably, the microparticle or particle is surrounded by an aqueous formulation (341) such as water and a stabilizer.

In certain preferred aspects, such as in a water/oil (w/o) emulsion or micro-emulsion, the active agent (e.g., DNA) is in the aqueous phase. The ABM (e.g., a cationic lipid) resides at the interface with the hydrophilic end complexed with DNA through, for example, an ionic interaction, and the lipophilic end immersed and/or dissolved and/or embedded in an immiscible phase (e.g., an oil phase). As used herein, the term “immiscible” pertains to phases that cannot mix to form a homogeneous mixture. In certain preferred embodiments, an encapsulation material, such as a hydrophobic polymer (e.g., PLGA) is also dissolved in the oil phase. Other suitable encapsulation materials include for example, surfactants, hydrophilic polymers, and micelles. Those of skill in the art will know of other encapsulation material suitable for use in the present invention. Without being bound by any particular theory, it is believed that the ABM holds the active agent through the emulsion process, and thus enhances encapsulation efficiency. The lipophilic end of the ABM faces outward and is able to make the particle (e.g., microparticle) smaller in size.

In certain aspects, the ABM “holds” the active agent and prevents or retards diffusion by for example, electrostatic interaction (e.g., ionic interaction), structural anchoring, molecular docking, hydrophobic interactions, adsorption, π-π interactions, Van der Waals forces or a combination thereof. In certain preferred aspects, electrostatic interaction can be employed for use in w/o type microparticles, while structural anchoring, and adsorption can be used for w/o, or o/w (i.e., the active agent can be hydrophilic or lipophilic). Hydrophobic interactions are preferably used for o/w type emulsions.

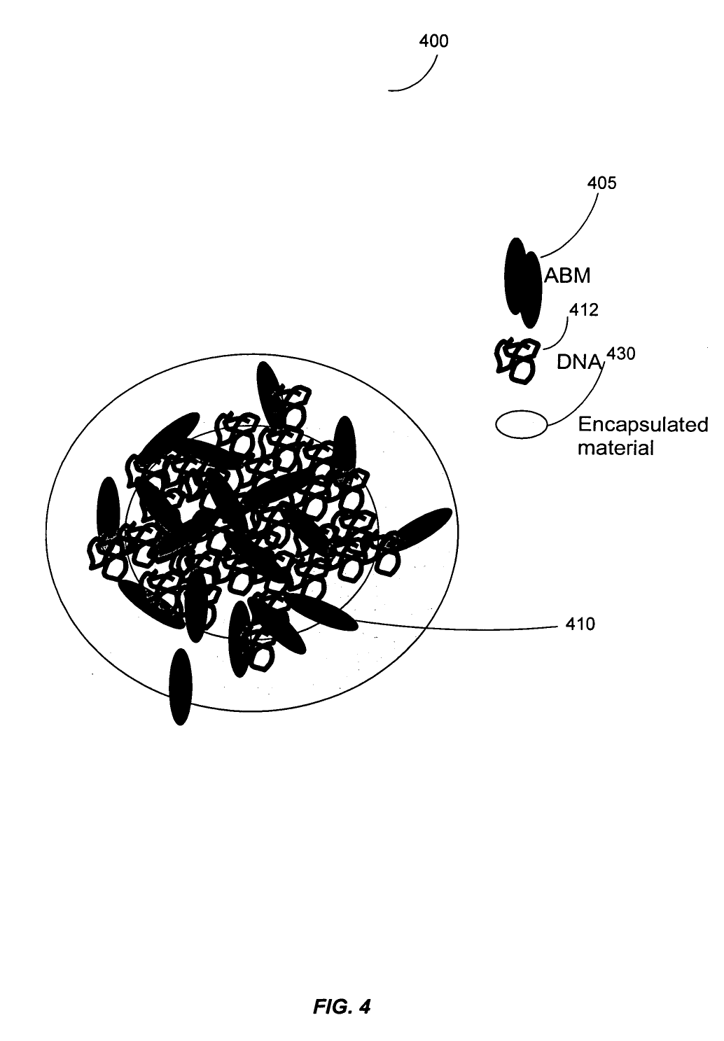

FIG. 4 is a diagram of a representative embodiment of a delivery particle (400) of the present invention. This diagram is merely an illustration and should not limit the scope of the claims herein. One of ordinary skill in the art will recognize other variations, modifications, and alternatives.

As shown therein, in one embodiment, the delivery particle comprises an inner core (410) which is solid material comprising “largely” ABM (405), DNA (412) and some encapsulation material (430). Preferably, the inner core is a DNA-rich mixed phase. The outer layer (e.g., the annular region) is preferably a polymer-rich region comprising mainly the encapsulation material. In certain aspects, the DNA in the inner core can be an aggregate, so it is possible that DNA is “dispersed” in the encapsulation material.

As such, the present invention provides a delivery particle, comprising: an inner core having an active agent; an amphiphilic binding molecule; and a polymeric outer layer, wherein the amphiphilic binding molecule is situated between the inner core and the outer layer. Alternatively, the inner core contains an aqueous media. In certain aspects, if DNA is aggregated, such that it floats in a solid or liquid media, the DNA is referred to as being “dispersed” within the media. Alternatively, if DNA is aggregated “without media,” the DNA is in a neat phase.

In certain embodiments, the compositions and methods of the present invention produce a delivery microparticle having a homogeneous size distribution. Typical particle size distributions range from about 0.01 μm to about 1000 μm, preferably from about 0.1 μm to about 100 μm, more preferably from about 0.1 μm to about 50 μm, and most preferably from about 0.5 μm to about 10 μm in diameter.

The present invention can produce, for example, 1 μm sized particles, which are relatively monodisperse in size. By producing a microparticle that has a well defined and less variable size, the properties of the microparticle, such as when used for release of an active agent, can be better controlled. Thus, the present invention permits improvements in the preparation of sustained release formulations, controlled release formulations, or modified release formulations for administration to subjects.

A. Active Agents

A wide range of active agents can be employed in the present invention, such as nucleic acid, proteins, small molecules and various agents in whole or in part. Preferably, the active agent is incorporated into the microparticle during formation of the microparticle. In one embodiment, hydrophobic active agents can be incorporated into the organic solvent, while nucleic acid and hydrophilic active agents can be added to an aqueous component.

In certain aspects, the active agent is present in a range of about 0.002% to about 50% w/w, preferably about 0.01% to about 20% w/w of the encapsulation material used. In a preferred aspect, the active agent is present in a range of about 0.01% to about 10% w/w, such as about 7-8% w/w of the encapsulation material.

1. Nucleic Acids

In certain preferred aspects, the active agent is nucleic acid (e.g., DNA). The nucleic acid of interest can encode any protein. Nucleic acids of interest may encode, for example, enzymes, growth hormones, clotting factors, lysosomal enzymes, plasma proteins, plasma protease inhibitors, proteases, protease inhibitors, hormones, pituitary hormones, growth factors, somatomedins, gonadotrophins, apolipoproteins, insulinotrophic hormones, immunoglobulins, chemotactins, chemokines, interleukins, interferons, cytokines, fusion proteins, and antigens, such as, for example, viral antigens, bacterial antigens, fungal antigens, parasitic antigens, or antigens overexpressed on neoplastic cells.

In some embodiments of the present invention, the mammalian subject has a condition which is amenable to treatment or prevention by expression or over-expression of a protein which is normally present in a healthy mammalian subject. For example, the methods of the present invention may also be used to enhance expression of a protein present in a normal mammal, or to express a protein not normally present in a normal mammal, in order to achieve a desired effect (e.g., to enhance a normal metabolic process or to induce an immune response). In one aspect of the invention, the nucleic acid is expressed in intestinal epithelial cells. In other aspects of the invention, the nucleic acid is expressed in cells that are not intestinal epithelial cells, but cells that reside within the intestine either temporarily or permanently.

In an exemplary embodiment, the methods of the present invention can be used to treat a mammalian subject with an autoimmune disease by delivering a nucleic acid encoding a therapeutic protein to the gastrointestinal tract of the subject (e.g., delivery of a nucleic acid encoding interferon-β to the gastrointestinal tract to treat multiple sclerosis). In another exemplary embodiment, the methods of the present invention can be used to treat a mammalian subject having an inherited or acquired disease associated with a specific protein deficiency (e.g., diabetes, hemophilia, anemia, severe combined immunodeficiency). Such protein deficient states are amenable to treatment by replacement therapy, i.e., delivery of a nucleic acid to the gastrointestinal tract and expression of the encoded protein in the bloodstream to restore blood stream levels of the protein to at least normal levels. Secretion of a therapeutic protein to the gastrointestinal tract (e.g. by secretion of the protein into the saliva, pancreatic juices, bile, or other mucosal secretion) is appropriate where, for example, the subject suffers from a protein deficiency associated with absorption of nutrients (e.g. deficiency in intrinsic factor) or digestion (e.g., deficiencies in various pancreatic enzymes).

The methods of the present invention can also be used to treat a mammalian subject with a neoplastic disorder. Delivery of nucleic acids encoding antigens differentially overexpressed on the surface of neoplastic cells can be used to induce an immune response against such antigens and consequently against the neoplastic cells. Exemplary cancer antigens include, for example, HPV L1, HPV L2, HPV E1, HPV E2, PSA, placental alkaline phosphatase, AFP, BRCA1, Her2/neu, CA 15-3, CA 19-9, CA-125, CEA, hCG, urokinase-type plasminogen activator (uPA), plasminogen activator inhibitor, and MAGE-1.

The nucleic acid of interest is typically from the same species as the mammalian subject to be treated (e.g., human to human), but this is not an absolute requirement. Nucleic acid obtained from a species different from the mammalian subject can also be used, particularly where the amino acid sequences of the proteins are highly conserved and the xenogeneic protein is not highly immunogenic so as to elicit a significant, undesirable antibody response against the protein in the mammalian host.

The diseases and disorders to be prevented or treated include, but are not limited to, autoimmune disorders, blood disorders, cardiovascular disorders, central nervous system disorders, gastrointestinal disorders, metabolic disorders, neoplastic diseases, pulmonary disorders, and bacterial and viral diseases. Autoimmune disorders that can be treated according to the methods of the present invention include, for example, multiple sclerosis, arthritis, diabetes, systemic lupus erythematosus, and Grave's disease. Blood disorders that can be treated according to the methods of the present invention include, for example, anemia sickle cell anemia, a globin disorder, and a clotting disorder such as hemophilia. Cardiovascular disorders that can be treated or prevented according to the methods of the present invention include, for example, high blood pressure, high cholesterol, and angina. Central nervous system disorders that can be treated according to the methods of the present invention include, for example, Parkinson's disease, Alzheimer's disease, multiple sclerosis, and Lou Gehrig's disease. Gastrointestinal disorders that can be treated according to the methods of the present invention include, for example, esophageal reflux, lactose deficiency, defective vitamin B12 absorption, and inflammatory bowel disease (IBD). Metabolic disorders that can be treated according to the methods of the present invention include, for example, enzyme deficiencies, obesity, lysosomal storage disease, Hurler's disease, Scheie's disease, Hunter's disease, Sanfilippo diseases, Morqio diseases, Maroteaux-Lamy disease, Sly disease, and dwarfism. Neoplastic diseases that can be treated or prevented according to the methods of the present invention include, for example, colon cancer, stomach cancer, liver cancer, pancreatic cancer, lung cancer, breast cancer, skin cancer, leukemia, lymphoma, and myeloma. Pulmonary disorders that can be treated according to the methods of the present invention include, for example, cystic fibrosis, emphysema, and asthma.

Exemplary nucleic acids of interest include, but are not limited to, nucleic acid sequences encoding interferon β, interferon α, interferon γ, insulin, growth hormone, clotting factor VIII, clotting factor IX, intrinsic factor, and erythropoietin. Of particular interest is protein therapy in a mammalian subject (e.g., a bovine, canine, feline, equine, or human subject, preferably a bovine or human subject, more preferably a human subject) by expression of a nucleic acid encoding a protein (e.g., interferon β, insulin, growth hormone, clotting factor VIII, or erythropoietin) in a transformed mammalian cell. Preferably, the subject is a human subject and the nucleic acid expressed encodes a human protein (e.g., human insulin, human growth hormone, human clotting factor VIII, or human erythropoietin). Table 1 provides a list of exemplary proteins and protein classes which can be delivered by the methods of the present invention.

TABLE 1
SPECIFIC EXEMPLARY PROTEINS

| | |
|---|---|
| α-galactosidase | α-glucosidase, |
| glucocerebrosidase | β-glucuronidase |
| epidermal growth factor (EGF) | phenylalanine ammonia lyase |
| lipid-binding proteins (lbp) | apolipoprotein B-48 |
| apolipoprotein Al₂ | vasoactive intestinal peptide (VIP) |
| insulin | interferon-α2B |
| glucagon | interferon β |
| glucagon-like peptide (GLP) | |
| human growth hormone (hGH) | transforming growth factor (TGF) |
| erythropoietin (EPO) | ciliary neurite transforming factor (CNTF) |
| clotting factor VIII | insulin-like growth factor-1 (IGF-1) |
| bovine growth hormone (BGH) | granulocyte macrophage colony stimulating |
| | factor (GM-CSF) |
| platelet derived growth factor (PDGF) | interferon-α2A |
| clotting factor IX | |
| antithrombin III | brain-derived neurite factor (BDNF) |
| thrombopoietin (TPO) | insulintropin |
| IL-1 | tissue plasminogen activator (tPA) |
| IL-2 | urokinase |
| IL-1 RA | tumor necrosis factor alpha (TNF-α) |
| soluble CD4 | tumor necrosis factor beta (TNF-β) |
| IL-4 | somatostatin |
| IL-5 | purine nucleotide phosphorylase |
| IL-10 | α-1-antitrypsin |
| IL-12 | streptokinase |
| superoxide dismutase (SOD) | adenosine deamidase |
| catalase | calcitonin |
| fibroblast growth factor (FGF) (acidic or | arginase |
| basic) | |
| neurite growth factor (NGF) | phenylalanine ammonia lyase |
| granulocyte colony stimulating factor (G- | γ-interferon |
| CSF) | |
| L-asparaginase | pepsin |
| uricase | trysin |
| chymotrypsin | elastase |
| carboxypeptidase | lactase |
| sucrase | intrinsic factor |
| calcitonin | parathyroid hormone(PTH)-like hormone |
| Ob gene product | cholecystokinin (CCK) |
| gastric inhibitory peptide (GIP) | insulinotrophic hormone |
| enodthelian | transforming growth factor beta (TGF-β) |

EXEMPLARY CLASSES OF PROTEINS

| | |
|---|---|
| proteases | pituitary hormones |
| protease inhibitors | growth factors |
| cytokines | somatomedin |
| chemokines | immunoglobulins |
| gonadotrophins | interleukins |
| chemotactins | interferons |
| lipid-binding proteins | growth hormones |
| clotting factors | lysosomal enzymes |
| plasma proteins | plasma protease inhibitors |
| apolipoproteins | fusion proteins |
| antigens (e.g., viral antigens, bacterial antigens, | |
| fungal antigens, parasitic antigens, or | |
| antigens overexpressed on neoplastic cells) | |

In other embodiments of the present invention, the mammalian subject has a condition which is amenable to treatment or prevention by expression of a protein that is foreign to the mammalian subject. For example, delivery of a nucleic acid encoding a protein that is foreign to the mammalian subject can be used to generate an immune response against the protein. The nucleic acid can be expressed by, e.g., cells residing in the intestine, specifically, intestinal epithelial cells. In some embodiments, the protein encoded by the nucleic acid is secreted into the bloodstream. The methods of the invention can be used to treat or prevent viral infections (e.g., human immunodeficiency virus (HIV), Epstein-Barr virus (EBV), herpes simplex virus (HSV)), bacterial infections, fungal infections, and/or parasitic infections. Bacterial diseases that can be treated or prevented according to the methods of the present invention include, for example, diphtheria, Lyme disease, meningitis, food poisoning, and pneumonia. Viral diseases that can be treated or prevented according to the methods of the present invention include, for example, HIV, Epstein Barr virus, herpes simplex virus, hepatitis A, hepatitis B, hepatitis, C, hepatitis E, mumps, measles, polio, and chicken pox.

Bacterial antigens may be derived from, for example, Staphylococcus aureus, Staphylococcus epidermis, Helicobacter pylori, Streptococcus bovis, Streptococcus pyogenes, Streptococcus pneumoniae, Listeria monocytogenes, Mycobacterium tuberculosis, Mycobacterium leprae, Corynebacterium diphtheriae, Borrelia burgdorferi, Bacillus anthracis, Bacillus cereus, Clostridium botulinum, Clostridium difficile, Salmonella typhi, Vibrio chloerae, Haemophilus influenzae, Bordetella pertussis, Yersinia pestis, Neisseria gonorrhoeae, Treponema pallidum, Mycoplasm sp., Neisseria meningitidis, Legionella pneumophila, Rickettsia typhi, Chlamydia trachomatis, and Shigella dysenteriae. Viral antigens may be derived from, for example, human immunodeficiency virus (HIV), human papilloma virus, Epstein Barr virus, herpes simplex virus, human herpes virus, rhinoviruses, cocksackieviruses, enteroviruses, hepatitis A, hepatitis B, hepatitis C, hepatitis E, rotaviruses, mumps virus, rubella virus, measles virus, poliovirus, smallpox virus, influenza virus, rabies virus, and Varicella-zoster virus. Fungal antigens may be derived from, for example, Tinea pedis, Tinea corporus, Tinea cruris, Tinea unguium, Cladosporium carionii, Coccidioides immitis, Candida sp., Aspergillus fumigatus, and Pneumocystis carinii. Parasite antigens may be derived from, for example, Giardia lamblia, Leishmania sp., Trypanosoma sp., Trichomonas sp., Plasmodium sp., and Schistosoma sp.

The nucleic acids of interest are typically produced by recombinant DNA methods (see, e.g., Ausubel, et al. ed. (2001) Current Protocols in Molecular Biology). For example, the DNA sequences encoding the immunogenic polypeptide can be assembled from cDNA fragments and short oligonucleotide linkers, or from a series of oligonucleotides, or amplified from cDNA using appropriate primers to provide a synthetic gene which is capable of being inserted in a recombinant expression vector (i.e., a plasmid vector or a viral vector) and expressed in a recombinant transcriptional unit. Once the nucleic acid encoding an immunogenic polypeptide is produced, it may be inserted into a recombinant expression vector that is suitable for in vivo expression. Any technique known in the art may be used to isolate and amplify the nucleic acids of the present invention.

For eukaryotic expression (e.g., in an intestinal epithelial cell or a secretory gland cell), the construct may comprise at a minimum a eukaryotic promoter operably linked to a nucleic acid operably linked to a polyadenylation sequence. The polyadenylation signal sequence may be selected from any of a variety of polyadenylation signal sequences known in the art, such as, for example, the SV40 early polyadenylation signal sequence. The construct may also include one or more introns, which can increase levels of expression of the nucleic acid of interest, particularly where the nucleic acid of interest is a cDNA (e.g., contains no introns of the naturally-occurring sequence). Any of a variety of introns known in the art may be used.

The promoter used to direct expression of a heterologous nucleic acid depends on the particular application. The promoter is preferably positioned about the same distance from the heterologous transcription start site as it is from the transcription start site in its natural setting. As is known in the art, however, some variation in this distance can be accommodated without loss of promoter function. Suitable promoters include strong, eukaryotic promoter such as, for example, promoters from cytomegalovirus (CMV), mouse mammary tumor virus (MMTV), Rous sarcoma virus (RSV), and adenovirus. More specifically, suitable promoters include the promoter from the immediate early gene of human CMV (Boshart et al., Cell 41:521 (1985)) and the promoter from the long terminal repeat (LTR) of RSV (Gorman et al., Proc. Natl. Acad. Sci. USA 79:6777 (1982)).

Tissue specific promoters maybe used in the methods of the present invention. One of skill in the art will appreciate that any tissue specific promoter known in the art may be used, including, for example, intestine-specific promoters, secretory gland-specific promoters, muscle-specific promoters (see, e.g., Hoggatt et al., Circ. Res. 91(12):1151-9 (2002)), lung-specific promoters (see, e.g., Carr et al., J. Biol. Chem. (2003), available at http://wwwjbc.org/cgi/reprint/M300319200v1.pdf), liver-specific promoters, pancreas-specific promoters (see, e.g., Hansen et al., J. Clin. Invest. 110(6):827-33 (2002)), brain-specific promoters (see, e.g., Timmusk et al., Neuroscience 60(2):287-91 (1994)), kidney-specific promoters (see, e.g., Chiu et al., Prog. Nucleic Acid Res. Mol. Biol. 70:155-74 (2001)), mammary gland-specific promoters (see, e.g. U.S. Pat. No. 5,565,362), and prostate gland-specific promoters (see, e.g., Shirakawa et al., Mol. Urol. 4(2):73-82 (2000) and van der Poel et al. Cancer Gene Ther. 8(12):927-35 (2001)). Intestine-specific promoters may be used in accordance with the present invention and include, for example, villin promoters, FABP promoters, L-FABP promoters, iFABP promoters, surcrase-isomaltase promoters, and lactase-phlorizin hydrolase promoters. Secretory gland specific promoters may also be used in accordance with the present invention and include, for example, salivary α-amylase promoters and mumps viral gene promoters which are specifically expressed in salivary gland cells. Multiple salivary α-amylase genes have been identified and characterized in both mice and humans (see, for example, Jones et al., Nucleic Acids Res., 17(16):6613 (1989); Pittet et al., J. Mol. Biol. 182:359 (1985); Hagenbuchle et al., J. Mol. Biol., 185:285 (1985); Schibler et al., Oxf. Surv. Eukaryot. Genes 3:210 (1986); and Sierra et al., Mol. Cell. Biol., 6:4067 (1986) for murine salivary α-amylase genes and promoters; Samuelson et al., Nucleic Acids Res., 16:8261 (1988); Groot et al., Genomics, 5:29 (1989); and Tomita et al., Gene, 76:11 (1989) for human salivary α-amylase genes and their promoters). The promoters of these α-amylase genes direct salivary gland specific expression of their corresponding α-amylase encoding DNAs. These promoters may thus be used in the constructs of the present invention to achieve salivary gland-specific expression of a nucleic acid of interest. Sequences which enhance salivary gland specific expression are also well known in the art (see, for example, Robins et al., Genetica 86:191 (1992)).

Other components of the construct may include, for example, a marker (e.g., an antibiotic resistance gene (e.g., an ampicillin resistance gene or a hygromycin resistance gene) to aid in selection of cells containing and/or expressing the construct, an origin of replication for stable replication of the construct in a bacterial cell (preferably, a high copy number origin of replication), a nuclear localization signal, or other elements which facilitate production of the nucleic acid construct, the protein encoded thereby, or both.

In addition to the promoter, the expression vector typically contains a transcription unit or expression cassette that includes all the additional elements required for the expression of the nucleic acid in host cells. A typical expression cassette thus contains a promoter operably linked to the nucleic acid sequence and signals required for efficient polyadenylation of the transcript, ribosome binding sites, and translation termination. The nucleic acid sequence may typically be linked to a cleavable signal peptide sequence to promote secretion of the encoded protein by the transformed cell. Such signal peptides would include, among others, the signal peptides from tissue plasminogen activator, insulin, and neuron growth factor, and juvenile hormone esterase of Heliothis virescens. Additional elements of the cassette may include enhancers and, if genomic DNA is used as the structural gene, introns with functional splice donor and acceptor sites.

In addition to a promoter sequence, the expression cassette may also contain a transcription termination region downstream of the structural gene to provide for efficient termination. The termination region may be obtained from the same gene as the promoter sequence or may be obtained from different genes.

2. Small Molecules and Drugs

In certain aspects, the therapeutic agents, which are administered using the present invention, can be any of a variety of drugs, which are selected to be an appropriate treatment for the disease to be treated. Table 2 sets forth various small molecules suitable for use in the present invention.

TABLE 2
Exemplary Drug Classes and Drug

| Class of Therapeutic | Specific Examples |
|---|---|
| antineoplastic agents | vincristine, doxorubicin, mitoxantrone, |
| | camptothecin, cisplatin, bleomycin, |
| | cyclophosphamide, methotrexate, streptozotocin |
| antitumor agents | actinomycin D, vincristine, vinblastine, cystine |
| | arabinoside, anthracyclines, alkylative agents, |
| | platinum compounds, taxol |
| antimetabolites | |
| nucleoside analogs | methotrexate, purine, pyrimidine analogs. |
| anti-infective agents | |
| local anesthetics | dibucaine, chlorpromazine |
| β-adrenergic blockers | propranolol, timolol, labetolol |
| antihypertensive | clonidine, hydralazine |
| agents | |
| anti-depressants | imipramine, amitriptyline, doxepim |
| anti-conversants | phenytoin |
| antihistamines | diphenhydramine, chlorphenirimine, |
| | promethazine |
| antibiotic/antibacterial | gentamycin, ciprofloxacin, cefoxitin |
| agents | |
| antifungal agents | miconazole, terconazole, econazole, isoconazole, |
| | butaconazole, clotrimazole, itraconazole, nystatin, |
| | naftifine, amphotericin B |
| antiparasitic agents | |
| hormones | estrogen, testosterone, androgen, leuprolide |
| hormone antagonists | |
| immunomodulators | |
| neurotransmitter | |
| antagonists | |
| antiglaucoma agents | |
| vitamins | vitamin A, vitamin D |
| narcotics | morphine, |
| imaging agents | |
| non-steroidal | aspirin, indomethacin |
| anti-inflammatory | |
| drugs (NSAIDs) | |
| volume expander | serum albumin |

B. Amphiphilic Binding Molecules

In certain preferred aspects, the amphiphilic binding molecule (ABM) is, for example, a molecule with dual functionalities, or opposite functional properties on the molecule, such as at each end of the molecule. Opposite/dual functional properties include for example, hydrophobic/hydrophilic functional properties; positively charged/negatively charged functionality and the like. In certain aspects, the amphiphilic molecule is a cationic lipid. The term “cationic lipid” refers to any of a number of lipid species, which carry a net positive charge at a selective pH, such as physiological pH.

Suitable cationic lipids include, but are not limited to, N,N-dioleyl-N,N-dimethylammonium chloride (“DODAC”), N-(2,3-dioleyloxy)propyl)-N,N,N-trimethylammonium chloride (“DOTMA”), N,N-distearyl-N,N-dimethylammonium bromide (“DDAB”), N-(2,3-dioleoyloxy)propyl)-N,N,N-trimethylammonium chloride (“DOTAP”), 1,2-dimyristoyl-sn-glycero-3-trimethylammonium-propane (“DMTAP”), 1,2-dipalmitoyl-sn-glycero-3-trimethylammonium-propane (“DPTAP”), and 1,2-distearoyl-sn-glycero-3-trimethylammonium-propane (“DSTAP”), 3-(N-(N′,N′-dimethylaminoethane)-carbamoyl)cholesterol (“DC-Chol”), N-(1,2-dimyristyloxyprop-3-yl)-N,N-dimethyl-N-hydroxyethyl ammonium bromide (“DMRIE”), 1,2-dilauroyl-P-O-ethylphosphatidylcholine (“E-DLPC”), 1,2-dimyristoyl-P-O-ethylphosphatidylcholine (“E-DMPC”), 1,2-dipalmitoyl-P-O-ethylphosphatidylcholine (“E-DPPC”), and mixtures thereof.

Other cationic lipids suitable for use in the present invention are disclosed in, for example, U.S. Pat. Nos. 5,527,928, 5,744,625, 5,892,071, 5,869,715, 5,824,812, 5,925,623, and 6,043,390. In addition to cationic lipids, other suitable ABMs include molecules such as a protein, a polypeptide, a polypeptide fragment, a carbohydrate, a dendrimer, a receptor, a hormone, a toxin, and an amphipathic lipid.

In one embodiment, the typical amount of an ABM in the formulations of the present invention are for example, about 0.1 to about 100 times the amount of active agent on a molar basis. In certain preferred aspects, the amount is about 0.1 to about 10 times the amount of active agent on a molar basis. In certain aspects, the weight: weight (w/w) ratio of ABM: DNA is about 1:100 to about 20:1, preferably about 0.5:12 to about 10:1. In certain preferred aspects, the weight: weight ratio of ABM: DNA is about 6:1.

While primary functions of the ABM (e.g., cationic lipid or conjugated cationic lipid) include increasing the encapsulation efficiency and controlling the particle size, the ABM may also be used to introduce other features to the surface of the particle. For example, if a PEG-lipid conjugate is added to the double emulsion formulation, the lipid moiety aligns at the middle organic phase and the PEG moiety aligns in the outer phase. After the solvents evaporate from the formulation, the lipid is embedded in the resultant particle and the PEG is on the surface. This method can be used to modify the surface of the particle with PEG, peptides, or small molecules that can be conjugated to a lipid.

C. Encapsulation Material

The present compositions and methods are based upon water-in-oil-in-water (w/o/w) emulsions. In certain aspects, the active agent is added to an organic solution containing an encapsulation material such as a polymer (e.g., a hydrophobic polymer or a hydrophilic polymer). Preferably, the hydrophobic polymer is used to generate a hydrophobic coating. The hydrophobic polymer is preferably a biocompatible material such as PVC, silicone or a polyester.

Suitable encapsulation materials include, but are not limited to, poly(lactid-co-glycolide), poly(lactic acid), poly(caprolactone), poly(glycolic-acid), poly(anhydrides), poly(orthoesters), poly(hydroxybutyric acid), poly(alkylcyanoacrylate), poly(lactides), poly(glycolides), poly(lactic acid-co-glycolic acid), polycarbonates, polyesteramides, poly(amino acids), polycyanoacrylates, poly(p-dioxanone), poly(alkylene oxalate), biodegradable polyurethanes, blends, polystyrene, polymethylmethacrylate, and mixtures thereof. Those of skill in the art will know of other chemical classes suitable for use in the present invention.

Typical concentrations of encapsulation material (e.g., polymer) are, for example, about 0.1 mg to about 500 mg per mL of organic solvent. In preferred aspects, typical concentrations of encapsulation material are, for example, about 0.1 mg to about 100 mg per mL of organic solvent.

D. Stabilizing Agents

In certain embodiments, the compositions and methods of the present invention optionally comprise a stabilizing agent. Suitable stabilizing agents include, but are not limited to, polyvinyl alcohol, methylcellulose, hydroxyethyl cellulose, hydroxypropylmethylcellulose, gelatin, a carbomer, a poloxamer, and combinations thereof. Those of skill in the art will know of other chemical classes suitable for use in the present invention.

The stabilizing agents increase the solubility of the composition components and facilitate microparticle generation by ensuring quality emulsions. In one embodiment, the typical amount of stabilizer used in the present invention is, for example, about 0.1% to about 20% w/v of the outer phase (e.g., water).

IV. Administration

A microparticle comprising an active agent (e.g., DNA) of interest may be administered by any suitable technique known, including, but not limited to, orally (e.g., in a gene pill platform), parenterally, transmucosally (e.g., sublingually or via buccal administration), topically, transdermally, rectally and via inhalation (e.g., nasal or deep lung inhalation). Parenteral administration includes, but is not limited to, intravenous, intraarterial, intraperitoneal, subcutaneous, intramuscular, intrathecal, and intraarticular. As a skilled person will readily recognize, any microparticle within any stage of the process of making, is suitable for administration, including, for example, with reference to FIG. 2, items (220), (230), (250) and combinations thereof.

For buccal and/or oral administration, the composition can be in the form of tablets or lozenges formulated in a conventional manner. For example, tablets and capsules for oral administration can contain conventional excipients such as binding agents (for example, syrup, accacia, gelatin, sorbitol, tragacanth, mucilage of starch or polyvinylpyrrolidone), fillers (for example, lactose, sugar, microcrystalline cellulose, maize-starch, calcium phosphate or sorbitol), lubricants (for example, magnesium stearate, stearic acid, talc, polyethylene glycol or silica), disintegrants (for example, potato starch or sodium starch glycolate), or wetting agents (for example, wetting agents). The tablets can be coated according to methods well known in the art. When the dosage unit form is a capsule, it may contain, in addition to materials of the above type, a liquid carrier. A syrup of elixir may contain the active compound sucrose as a sweetening agent, methyl- and propyl-parabens as preservatives, a dye, and flavoring, such as cherry or orange flavor. Any material used in preparing any dosage unit form should be pharmaceutically pure and substantially non-toxic in the amounts employed. In addition, the active compounds may be incorporated into sustained-release preparations and formulations.

For oral administration, the compositions of the present invention may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin, and potassium bicarbonate, dispersed in a dentifrice, or added in a therapeutically-effective amount to a composition that may include water, binders, abrasives, flavoring agents, foaming agents, and humectants. Alternatively, the compositions may be fashioned into a tablet or solution form that may be placed under the tongue or otherwise dissolved in the mouth.

The compositions can also be administered retroductally, such as by delivery into the lumen of a salivary gland duct. A “salivary gland” is a gland of the oral cavity which secretes saliva, including the glandulae salivariae majores of the oral cavity (the parotid, sublingual, and submandibular glands) and the glandulae salivariae minores of the tongue, lips, cheeks, and palate (labial, buccal, molar, palatine, lingual, and anterior lingual glands). Suitable methods of retroductal introduction of the composition to the salivary gland duct include, for example, cannulation or injection of the composition into the salivary gland duct using a syringe, cannula, catheter, or shunt. The type of syringe, cannula, catheter, or shunt used is not a critical part of the invention. One of skill in the art will appreciate that multiple types of syringes, cannulas, catheters, or shunts may be used to administer compositions according to the methods of the present invention.

Retroductal delivery of the composition using the methods of the present invention may be via gravity or an assisted delivery system. Suitable assisted delivery systems include metering pumps, controlled-infusion pumps and osmotic pumps. The particular delivery system or device is not a critical aspect of the invention. One of skill in the art will appreciate that multiple types of assisted delivery systems may be used to deliver compositions according to the methods of the present invention. Suitable delivery systems and devices are described in U.S. Pat. Nos. 5,492,534, 5,562,654, 5,637,095, 5,672,167, and 5,755,691. One of skill in the art will also appreciate that the infusion rate for delivery of the composition may be varied. Suitable infusion rates may be from about 0.005 mL/min to about 1 mL/minute, preferably from about 0.01 mL/min to about 0.8 mL/min., more preferably from about 0.025 mL/min. to about 0.6 mL/min. It is particularly preferred that the infusion rate is about 0.05 mL/min.

In one embodiment, when the DNA of interest is introduced using a microparticle of the present invention, one first determines in vitro the optimal values for the DNA:microparticle ratios and the absolute concentrations of DNA and lipid as a function of cell death and transformation efficiency for the particular type of cell to be transformed. These values can then be used in or extrapolated for use in in vivo transformation. The in vitro determinations of these values can be readily carried out using techniques which are well known in the art.

Preferably, the DNA construct contains a promoter to facilitate expression of the DNA of interest within a cell, such as a pancreatic cell, or salivary gland cell. Preferably, the promoter is a strong, eukaryotic promoter. Exemplary eukaryotic promoters include promoters from cytomegalovirus (CMV), mouse mammary tumor virus (MMTV), Rous sarcoma virus (RSV), and adenovirus. More specifically, exemplary promoters include the promoter from the immediate early gene of human CMV (Boshart et al., Cell 41:521-530, 1985) and the promoter from the long terminal repeat (LTR) of RSV (Gorman et al., Proc. Natl. Acad. Sci. USA 79:6777-6781, 1982). Of these two promoters, the CMV promoter is preferred as it provides for higher levels of expression than the RSV promoter. The DNA of interest may be inserted into a construct so that the therapeutic protein is expressed as a fusion protein (e.g., a fusion protein having β-galactosidase or a portion thereof at the N-terminus and the therapeutic protein at the C-terminal portion). Production of a fusion protein can facilitate identification of transformed cells expressing the protein (e.g., by enzyme-linked immunosorbent assay (ELISA) using an antibody which binds to the fusion protein).

It may also be desirable to produce altered forms of the therapeutic proteins that are, for example, protease resistant or have enhanced activity relative to the wild-type protein. Further, where the therapeutic protein is a hormone, it may be desirable to alter the protein's ability to form dimers or multimeric complexes. For example, insulin modified so as to prevent its dimerization has a more rapid onset of action relative to wild-type, dimerized insulin.

The construct containing the DNA of interest can also be designed so as to provide for site-specific integration into the genome of the target cell. For example, a construct can be produced such that the DNA of interest and the promoter to which it is operably linked are flanked by the position-specific integration markers of Saccharomyces cerevisiae Ty3. The construct for site-specific integration additionally contains DNA encoding a position-specific endonuclease, which recognizes the integration markers. Such constructs take advantage of the homology between the Ty3 retrotransposon and various animal retroviruses. The Ty3 retrotransposon facilitates insertion of the DNA of interest into the 5′ flanking region of many different tRNA genes, thus providing for more efficient integration of the DNA of interest without adverse effect upon the recombinant cell produced. Methods and compositions for preparation of such site-specific constructs are described in U.S. Pat. No. 5,292,662, incorporated herein by reference with respect to the construction and use of such site-specific insertion vectors.

### V. EXAMPLES

The following examples are offered to illustrate, but not to limit the present invention.

### Example I

An aqueous DNA solution (2 mg of plasmid DNA in 0.3 mL TE buffer) was added to a solution of polymer (50:50 PLG) in CH₂Cl₂ (6 mL) to form a water in oil (w/o) emulsion. This solution was emulsified by vortexing at 2500 rpm for 15 sec. DOTAP (12.5 mg) was added and the emulsion was mixed by vortexing (2500 rpm/15 sec.). The resulting emulsion was then added to an aqueous solution (8% PVA, 100 mL) to form a water in oil in water (w/o/w) emulsion. The solution was allowed to stir until the oil layer (CH₂Cl₂) evaporated, resulting in a particle that encapsulated the inner water (DNA) layer. The particles were collected by centrifuging (1500 rpm, 15 min.). The supernatant was decanted and the particles were washed with 70 mL of water. This process was repeated and the microparticles were transferred to a 20 mL vial and lyophilized. The particles were then collected and stored at 0° C. Results indicated that this formulation increased the encapsulation efficiency of DNA and decreased particle size.

### Example II

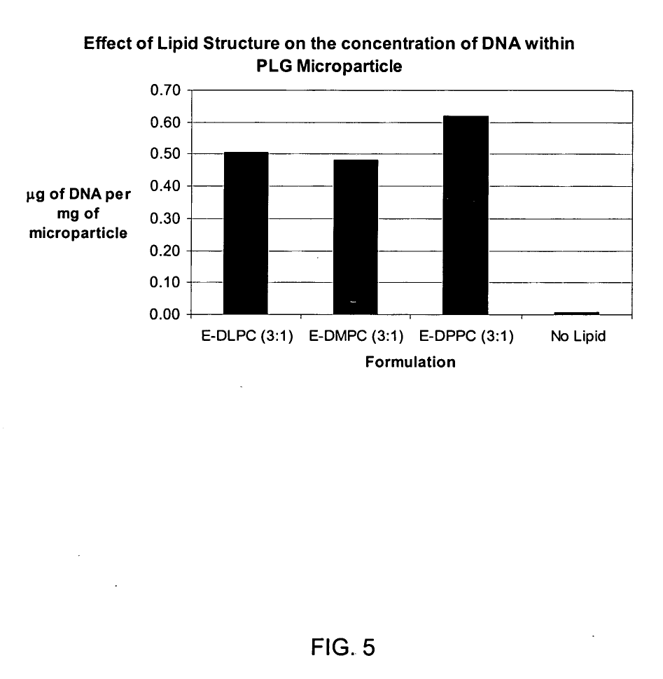

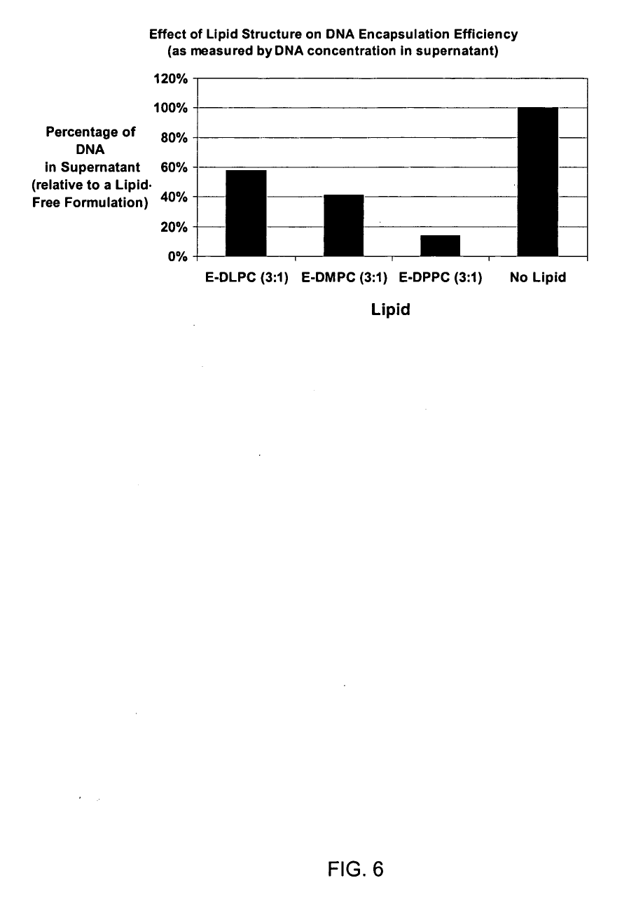

This example illustrates the effect of PLGA microparticles on the encapsulation efficiency of plasmid DNA. All microparticles were prepared with 25 mg of 50:50 poly(lactide-co-glycolide) and 250 μg of plasmid DNA. Three different lipids, E-DLPC, E-DMPC, and E-DPPC, were added at a 3:1 charge ratio. The PLGA coating was dissolved in an organic solvent and then an aqueous detergent solution was added to disrupt any interaction between DNA and the cationic lipid (ABM). After the DNA was quantified using a Pico-Green assay (Molecular Probes), the concentration of DNA within the microparticles was determined by dividing the amount of DNA that was detected by the mass of the microparticle sample (FIG. 5). In a subsequent experiment (FIG. 6), the encapsulation efficiency was measured to determine the amount of DNA that was actually encapsulated during the formulation procedure. This parameter was calculated based upon the concentration of DNA that was detected in the supernatant and wash solutions from the microparticle preparation protocol. The relative amount of DNA found in the supernatant was expressed as a percentage of DNA found in the supernatant of a lipid-free formulation. Both of these experiments demonstrate that the encapsulation efficiency and DNA concentration are dependent upon the structure of the cationic lipid. As the length of the carbon chain in the hydrophobic domain of the cationic lipid increased, both of these parameters increased.

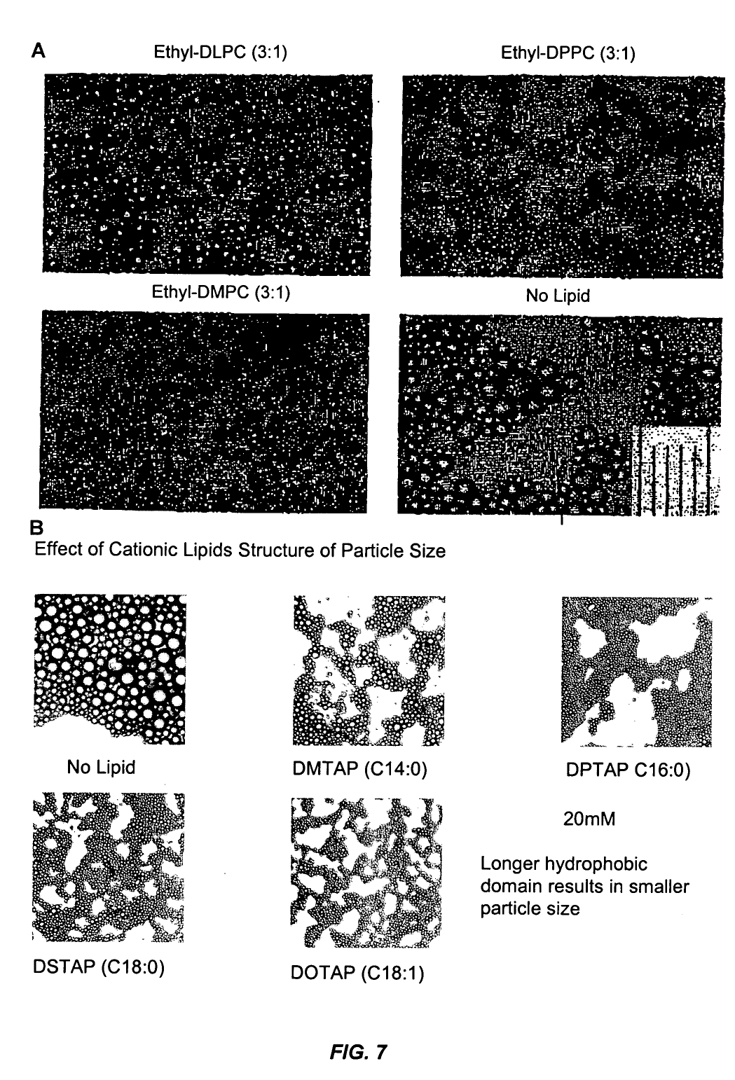

After the particles were purified, the particle size was determined using light microscopy. FIG. 7A depicts the particle size of different PLGA-cationic lipid (ABM) formulations under 400× magnification. These images demonstrate that the inclusion of a cationic lipid (ABM) into the formulation process results in a dramatic decrease in particle size. Moreover, the particle size is influenced by the chemical structure of the cationic lipid (ABM). The particle size decreases when cationic lipids with longer hydrophobic domains are used in the formulation.

### Example III

The effect of cationic lipid structure on encapsulation efficiency was determined by measuring the amount of DNA that remained in the supernatant/washes that were collected during the formulation process and the amount of DNA that was detected in the microparticles. The supernatant samples were prepared by diluting the supernatant samples with a 1% Zwittergent/TE buffer. The microparticle samples were analyzed by dissolving the microparticle coating with methylene chloride and then extracting the DNA with a 1% Zwittergent/TE buffer. The DNA concentration was determined using the Pico-Green reagents (Molecular Probes).

The encapsulation efficiency was calculated for three different cationic lipids, DMTAP, DPTAP, and DSTAP, at two charge ratios by multiplying the concentration of DNA in the particles by the mass of the particles collected and dividing the product by the amount of DNA initially added to the formulation (250 μg). The results are presented in Table 3 below.

TABLE 3
Analysis of DNA in supernatant and particles

| | | Amt of DNA | Amt of | μg of | |
|---|---|---|---|---|---|
| | | found in | DNA in | DNA/mg | |
| | Charge | Supernatant | Particle | of | Encapsulation |
| Lipid | Ratio | (μg) | (μg) | Particle | Efficiency |
| DMTAP | 2 | 24.23 | 77.71 | 3.89 | 31.09% |
| DMTAP | 4 | 26.49 | 225.29 | 11.26 | 90.12% |
| DPTAP | 2 | 28.51 | 157.07 | 7.85 | 62.83% |
| DPTAP | 4 | 15.57 | 86.46 | 4.32 | 34.58% |
| DSTAP | 2 | 37.64 | 185.74 | 9.29 | 74.30% |
| DSTAP | 4 | 3.74 | 125.87 | 6.29 | 50.35% |
| none | 0 | 201.56 | 0.24 | 0.01 | 0.09% |

Structure of Cationic Lipids Used:

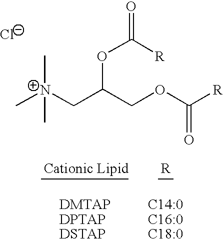

These results indicate the encapsulation efficiency is influenced by lipid structure.

After the particles were purified, the particle size was determined using light microscopy. FIG. 7B depicts the particle size of different PLGA-cationic lipid (ABM) formulations under 400× magnification. These images demonstrate that the inclusion of a cationic lipid (ABM) such as DMTAP, DPTAP, or DSTAP into the formulation process results in a dramatic decrease in particle size. Moreover, the particle size is influenced by the chemical structure of the cationic lipid (ABM). The particle size decreases when cationic lipids (ABM) with longer hydrophobic domains are used in the formulation.

### Example IV

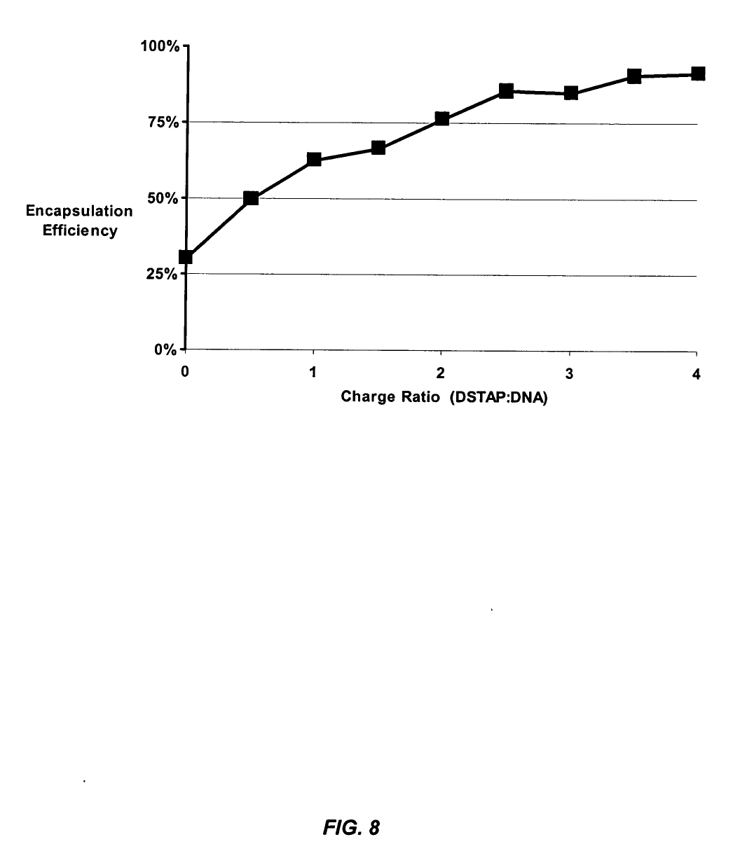

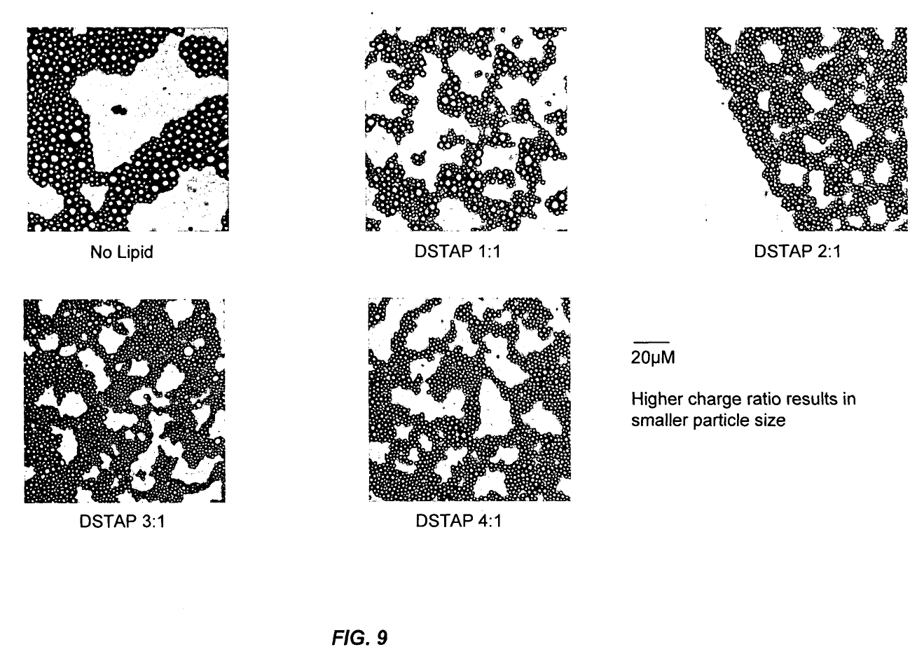

The effect of cationic lipid (ABM) concentration on encapsulation efficiency was determined for the cationic lipid DSTAP according to the experimental protocol of Example III. As shown in FIG. 8, higher DSTAP:DNA charge ratios, which correspond to increasing (ABM) cationic lipid concentration, resulted in higher DNA encapsulation efficiencies. Further, FIG. 9 illustrates that higher DSTAP:DNA charge ratios also resulted in smaller particle sizes. These particles were more homogenous and therefore displayed less polydispersity. The particles generated with DSTAP were approximately 1-3 μm in diameter, as compared to the larger and less homogenous population of particles generated in the absence of DSTAP (5-10 μm).

### Example V

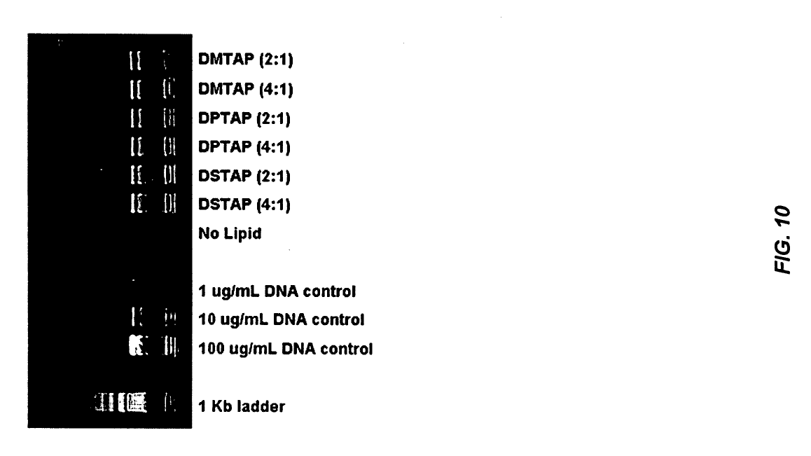

Many of the procedures that are used to formulate small molecule drugs are not amenable to large/more sensitive biopolymers, such as DNA, because of the excessive temperatures, high stir rates, etc. To determine the effect of the double emulsion method on DNA integrity, the DNA was extracted from DMTAP, DPTAP, or DSTAP-containing microparticles and then analyzed using agarose gel electrophoresis. As shown in FIG. 10, the DNA remains intact following formulation using the double emulsion technique in the presence of either DMTAP, DPTAP, or DSTAP. The ability of the cationic lipid to protect the DNA is independent of the cationic lipid structure or the cationic lipid:DNA ratio.

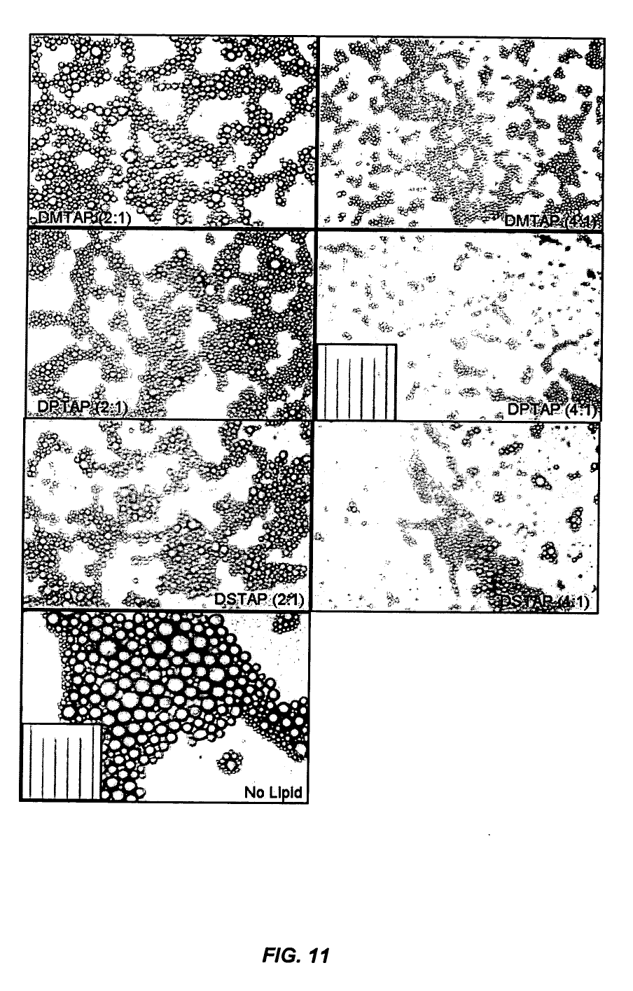

After the particles were purified, the particle size was determined using light microscopy. FIG. 11 depicts the particle size of different PLGA-cationic lipid (ABM) formulations under 400× magnification. These images demonstrate that the inclusion of the cationic lipid (ABM) DMTAP, DPTAP, or DSTAP into the formulation process resulted in a dramatic decrease in particle size. Moreover, the particle size is influenced by the chemical structure of the cationic lipid (ABM). As the chain length increases, the particle size decreases. Furthermore, increasing the cationic lipid:DNA ratio also produced smaller particles.

### Example VI

This example illustrates an in vitro analysis of microparticle transfection efficiency in CHO cells.

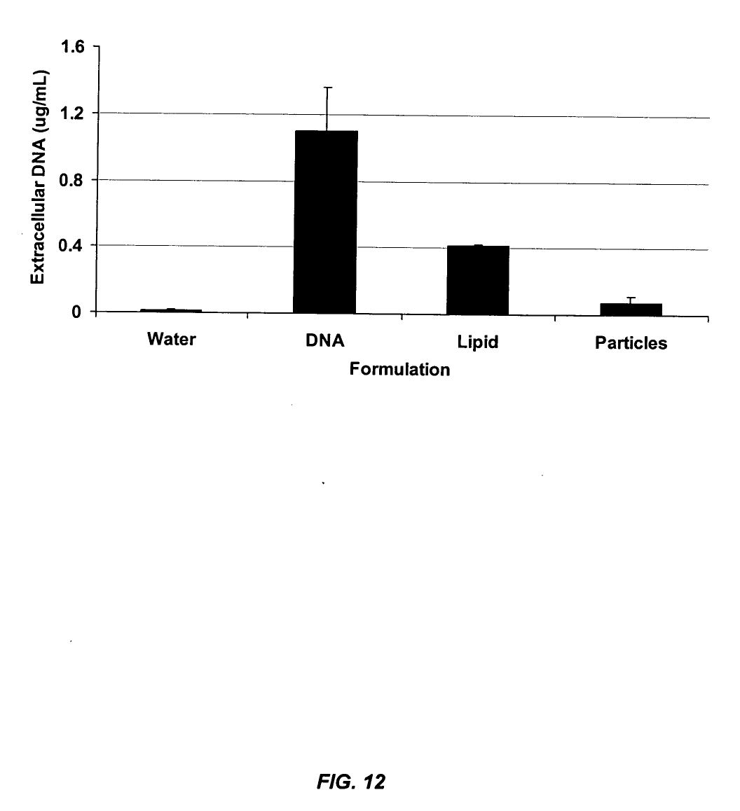

Microparticles containing plasmid DNA encoding secreted alkaline phosphatase (SEAP) were prepared as described in Example I. The cationic lipid (ABM) DSTAP was used in the microparticle formulation. The functionality of the plasmid DNA that was encapsulated in the microparticles was determined by treating CHO cells with four different formulations: water only, plasmid DNA in water, plasmid DNA in a DSTAP liposome, and plasmid DNA encapsulated in microparticles. These formulations were administered to CHO cells in the presence of fetal bovine serum (FBS). After 2 hours, all of the formulations were removed and the cells were treated with growth media. To analyze DNA uptake, the solution that was taken off of the cells after 2 hours was analyzed for DNA concentration (FIG. 12). The highest concentration of plasmid DNA was found in the plasmid DNA solution that was administered to CHO cells. Less DNA was detected in the other solutions.

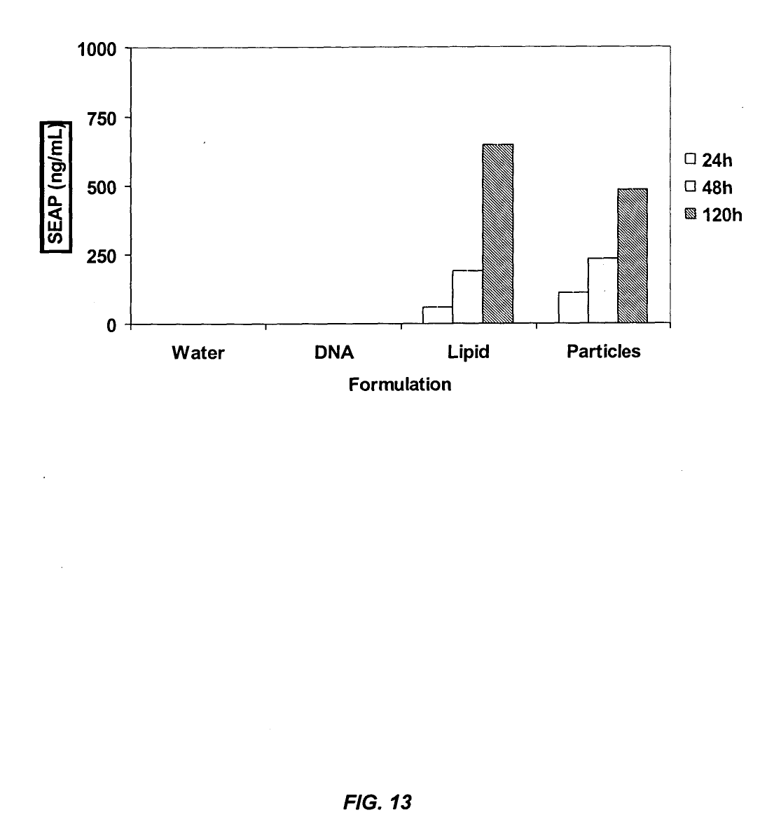

FIG. 13 shows the results of gene expression studies in CHO cells using the microparticles of the present invention. Both microparticle and control samples were tested by administering 100 μL (1 μg of DNA) to each well containing CHO cells in 100 μL of media. After 2 hours, the media was removed and replaced with 500 μL of serum positive media. At the indicated time points [24 h (white), 48 h (grey), and 120 h (hatched)], the media was removed, immediately frozen until analysis, and replaced with fresh media. While microscopic analysis of the cell population did not reveal any observable toxicity, the levels of SEAP expression from cells treated with DSTAP liposomes were similar to those of cells treated with the microparticle formulation, suggesting that the DNA is released from the microparticle formulation in a manner that is kinetically similar to a liposome formulation.

### Example VII

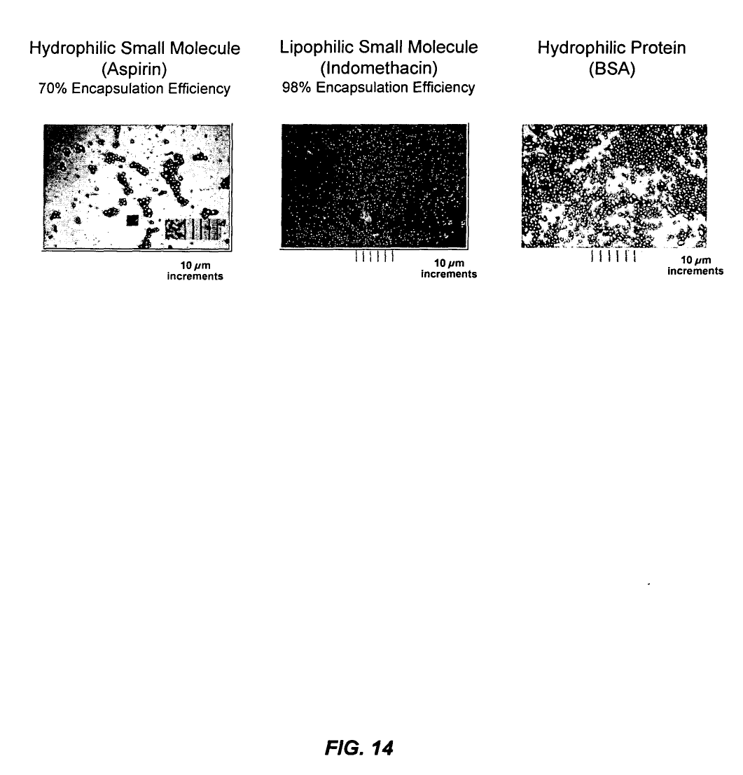

The particle size and encapsulation efficiency of microparticles containing pharmaceutically active ingredients other than plasmid DNA were determined. Small molecules such as aspirin and indomethacin were efficiently encapsulated into the microparticles of the present invention (70% and 98% encapsulation efficiency, respectively). As shown in FIG. 14, microparticles containing either of these small molecules were both homogeneous and small in size. Similar results were obtained with microparticles containing the hydrophilic protein bovine serum albumin (BSA). Thus, these data demonstrate that the double emulsion formulation process of the present invention can be applied to encapsulate and deliver other pharmaceutically active ingredients.

### Example VIII

This example illustrates antibody and T cell responses to antigens encoded by the DNA microparticle compositions of the present invention.

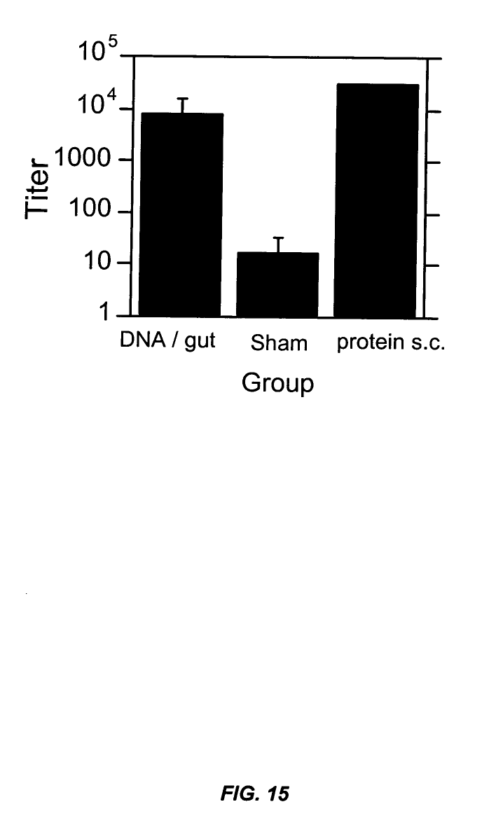

A mouse surgical model was used to simulate oral delivery of enteric coated DNA. After laparotomy, a needle was inserted through the intestinal wall and plasmid DNA was injected directly into the lumen of the duodenum. After several weeks, a significant antibody response that was specific to the protein encoded by the injected DNA was observed. Initial experiments used human growth hormone (hGH) as a model antigen because hGH is immunogenic in rodents. The average anti-hGH IgG titers exceeded 3.0×10⁴, and were comparable to those observed in mice treated with subcutaneous injection of hGH protein (FIG. 15).

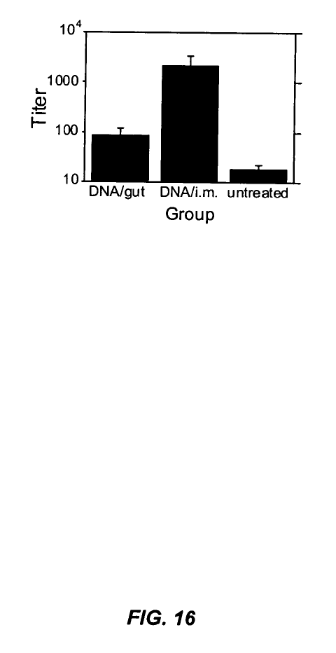

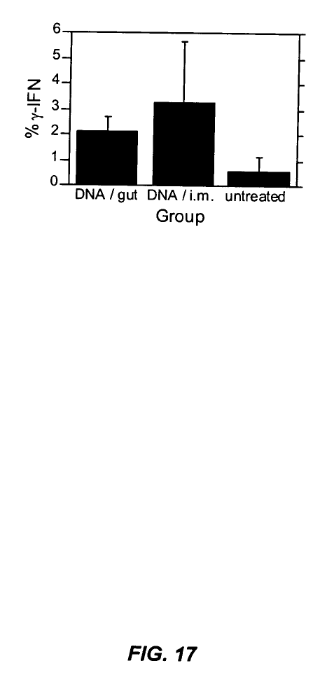

Injection of plasmid encoding HIV gp120 into the intestine also resulted in a significant antibody response against the protein product (FIG. 16). I.m. injection of gp120 DNA, included for comparison, has previously been shown to elicit strong immune responses in Balb/c mice. The immunodominant epitope recognized by Balb/c mice to HIV gp120 is composed of the V3 loop peptide (GPGRAFYTT) and MHC class I D^{d}. The ability of gene delivery to the intestine to induce a cytotoxic T cell response was evaluated by isolating splenocytes from intestinal, i.m., or unvaccinated mice and pulsing the splenocytes in culture with the immunodominant peptide. Peptide recognizing T cells produce intracellular γ-IFN, which was measured by flow cytometry. The average response between i.m. and intestinal vaccinated animals was similar (FIG. 17). This experiment demonstrates that DNA transfer to the intestines can promote cytotoxic T cell responses to the encoded antigen.

While direct administration of DNA to the small intestine provided some information about what is possible by oral DNA delivery, it is impractical in regard to a vaccine protocol. Ingestion of naked DNA will lead to DNA degradation by nucleases and the acidic environment of the stomach. In order to improve the survival of DNA for oral administration, the DNA was formulated as gastroprotective microspheres using cationic lipid (AMB) technology.

Microparticles were prepared using the w/o/w double emulsion process in the presence of cationic lipids to complex with the DNA and also serve as a hydrophobic barrier to improve DNA loading efficiency. Human growth hormone (hGH) plasmid DNA (2 mg) was dissolved in TE buffer (pH=7.4) and mixed with PLGA/dichloromethane solution (200 mg in 6 mL). The mixture was vortexed to form the first w/o emulsion. At this point, 1,2-Diphytanoyl-sn-Glycero-3-Phosphoethanolamine (at a 3:1 lipid to DNA charge ratio) was added to complex with the DNA. After 5 seconds of vortexing, the mixture was quickly poured into an aqueous solution containing 8% (w/v) aqueous PVA to form a w/o/w emulsion. The w/o/w emulsion was stirred at room temperature for 4 hours to evaporate the dichloromethane and form PLGA microparticles. Microparticles were then collected by centrifugation, followed by lyophilization. The solid particles were then suspended in orange-flavored gelatin prior to administration.

Mice were fed DNA microparticles contained within gelatin (DNA/gelatin), or gelatin alone (no DNA/gelatin) on weeks 0 and 3. DNA injected i.m. without gelatin (i.m.) served as a positive control and naïve mice served as negative controls. Antibody responses were measured in plasma on week 6 using an anti-hGH IgG ELISA. Animals that were fed the gelatin/DNA particles demonstrated a positive antibody response whereas animals that were fed no DNA/gelatin did not.

### Example IX

This example illustrates that pH sensitive polymers produce coated particles with enteric protecting materials.

Maximum loading efficiency is a key objective, and this parameter is largely controlled by the ABM. However, loading efficiency is also affected by composition of the particle shell. Two pH sensitive compounds [cellulose acetate phthalate (CAP) and Eudragit S-100] were evaluated in this system. In this process, CAP is mixed with PLGA in dichloromethane/isopropyl alcohol (10:1 volume ratio) as the oil phase of W/O emulsion before adding the ABM.

A second key objective for the enteric coat is uniformity coverage, with a target of 90% coating of each particle. The PLGA particle surface is coated by re-suspending particles in solvents that dissolve enteric coating material, but not PLGA. Silica was added to prevent the coated particles from clumping. Because enteric coating materials and biodegradable polymers have different solubility profiles and process tolerances, success with this system depends on the balance of materials and process.

The effectiveness of enteric coating is evaluated in vitro by a low pH challenge study. Enteric coated particles is suspended in a pH=1.2 (empty stomach) or pH=3.5 (full stomach) buffer for 10, 30, and 60 minutes, followed by buffer neutralization and extraction as follows: each sample (˜2 mg) is treated with 1 mL of methylene chloride and allowed to stir overnight, then extracted with 1% Zwittergent in Tris/EDTA buffer. To determine DNA concentration, the aqueous layer is diluted 1:9 with Tris/EDTA buffer (pH 8) and then quantified using Pico-Green reagent. DNA integrity is determined by agarose gel electrophoresis and visualization with ethidium bromide.

DNA release rate is adjusted by controlling the polymer:DNA ratio; which defines the thickness of the encapsulated shell. A lower polymer:DNA ratio will increase the release rate. Varying polylactide (PLA) to polyglycolide (PGA) ratio can also alter the release rate. Alternatively, incorporated disintegrants in PLGA matrix facilitate a faster release rate.

DNA release rate is evaluated using a dialysis method. Particles are confined in a 200 nm dialysis membrane and immersed in a neutral buffer solution to maintain a sink condition at all times. Samples are taken from the buffer solution at different time points (10, 30, and 60 minutes) to quantify DNA content as described above.

In one aspect, the ideal release rate profile is zero order for double emulsion process with all DNA released within 8 hours and no initial burst.

### Example X

This example describes construction of some of the plasmid DNAs that can be conveniently used for the tissue specific expression of interferon β (IFN-β) from the microparticles of the present invention.

Certain viral promoters produce a large quantity of protein for a short period of time, but the expression is ubiquitous and not restricted to the targeted tissues. In some circumstances, it may be desirable to use tissue-specific transcriptional elements so that protein is expressed in a cell type-specific manner.

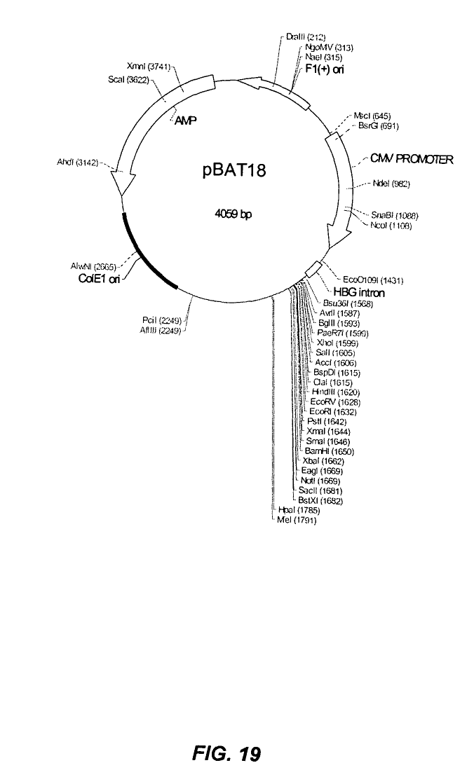

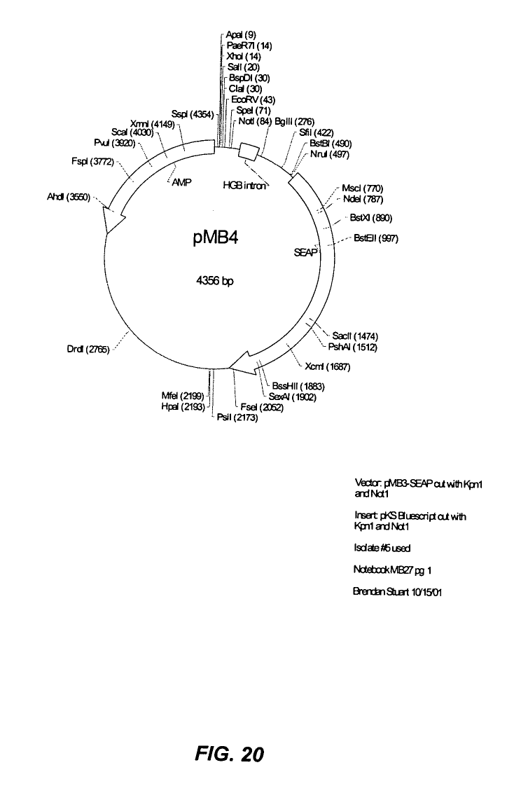

A novel plasmid (based on pBAT18, see FIG. 19 and SEQ ID NO:1) was constructed that has the CMV IE promoter cleanly deleted by PCR (pMB4, see FIG. 20 and SEQ ID NO:2). A cDNA encoding a protein of interest or the marker gene secreted alkaline phosphatase (SEAP) can be inserted into this plasmid to form a promoterless vector. Tissue-specific transcriptional elements can be rapidly cloned into these vectors and screened for transgene expression. For example, various promoters can be easily inserted into this plasmid to drive expression of a cDNA encoding SEAP or a protein of interest (e.g., IFN-β).

The plasmid pORF-IFN-β (Invivogen, Inc.), which contains the wild-type cDNA from IFN β, was subcloned into the mammalian expression vector pBAT18 by ligating the AgeI-NheI IFN-β fragment with pBAT18 digested with XmI-XbaI to form pBATh IFN-β.

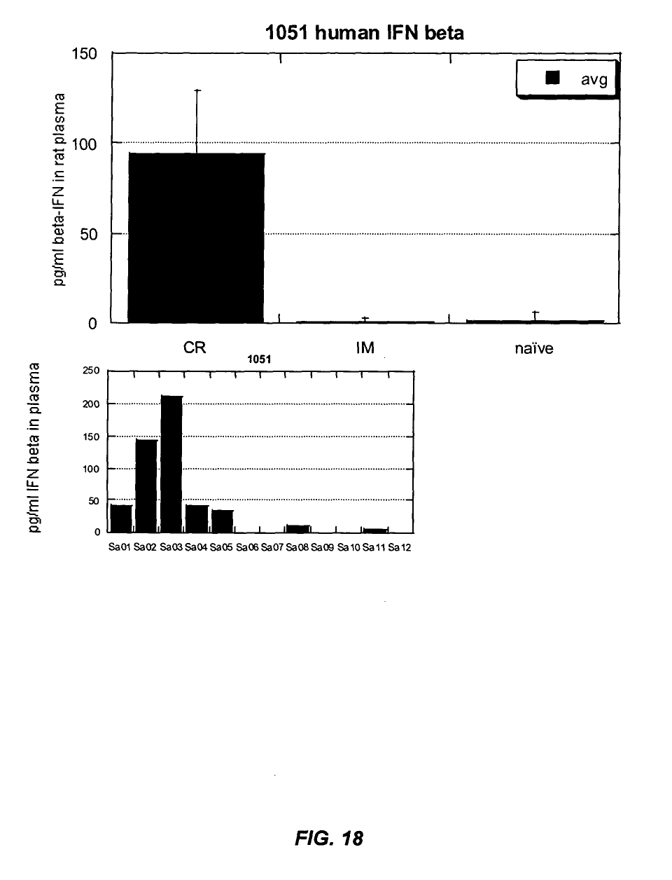

The pBAThIFNB construct was used to test the expression level of IFN-β. 175 μg plasmid DNA was formulated with Congo Red (CR) dye (6 mg/mL) and delivered retroductally to rat submandibular glands. Plasma samples were assayed with Biosource IFN-β ELISA kit, along with a protein standard curve. Delivery of IFN-β cDNA resulted in the protein being expressed and secreted in vivo. FIG. 18 shows that IFN-β is detectable by a standard protein assay known to those of skill in the art.

### Example XI

This example describes the in vitro testing of some of the plasmid DNAs that can conveniently be used for the expression of proteins in secretory gland and “gene pill” platforms.

A rapid in vitro expression screen can be carried out using tissue-specific promoters and secreted alkaline phosphatase (SEAP). For example, intestine-specific transcriptional elements can be screened. Suitable transcriptional elements for intestine-specific protein expression may include, for example, promoters for villin, FABP and iFABP, and α-Gal. The transcriptional elements may be tested in combination with other elements including viral and non-viral enhancer and 5′UTRs. Constructs containing the transcriptional elements can be transfected into the intestinal epithelial cell line, CaCO₂, and screened for expression and secretion of the marker protein SEAP. This method can conveniently be used to screen a number of transcriptional elements as well as combinations of transcriptional elements.

Once the transcriptional elements have been identified in vitro, the constructs can be tested in vivo using the delivery systems described herein. For example, IFN-β plasmid DNA constructs can be formulated in a gene pill platform and delivered orally to animal models. The gene pill can be used to target DNA to specific target tissues or cells, i.e., mammalian intestinal epithelial cells. Protein expression can be measured using any means known to those of skill in the art including, for example, sandwich ELISAs. Protein function can also be measured using any means known in the art. For example, a cytopathic effect inhibition assay can be used to measure the functionality of the IFN-β.

### Example XII

This example describes transfer of nucleic acids encoding therapeutic proteins (e.g., interferon β) using the DNA microparticles described herein.

Delivery of therapeutic proteins such as interferon β for treatment of diseases has substantial disadvantages, including, for example, poor dose control, poor bioavailability, and complicated manufacturing processes. These disadvantages can be circumvented using gene therapy. Gene therapy is an alternative treatment for many diseases that are currently treated with protein-based therapies. The delivery of genetic material (rather than a protein) simplifies the manufacturing process and provides an opportunity for better dose control. In addition, gene therapy using synthetic vectors may be safer than many protein-based therapies. Transfecting the cells of the gastrointestinal tract with a nucleic acid encoding a therapeutic protein (e.g., interferon β) provides a convenient method to introduce the therapeutic protein into the bloodstream and treat disease. Since the gastrointestinal tract readily degrades plasmid DNA, an efficient method for the delivery of nucleic acids to the gastrointestinal system is needed. To address these issues, a formulation comprising an encapsulating polymer, an amphiphilic binding molecule (ABM), and a nucleic acid encoding a therapeutic protein (e.g., interferon β) was developed. This formulation is designed to efficiently transfect the cells of the gastrointestinal system, resulting in expression of interferon β protein into the bloodstream and disease treatment.

Composition

A composition containing a nucleic acid encoding interferon β encapsulated in a particle comprising an encapsulating polymer and an amphiphilic binding molecule (ABM) was developed. The composition can be manufactured using any method known to those of skill in the art, including, for example, spray drying, co-acervation, double emulsion, solvent diffusion, freeze drying, and interfacial polymerization.

Delivery of Composition

The delivery of the composition into the gastrointestinal system results in the expression of interferon β. This composition is designed for oral administration and is capable of reaching the surface of the cells that line the gastrointestinal tract (e.g., intestinal epithelial cells) without compromising the functional integrity of the nucleic acid. The particle is capable of tolerating enduring high concentrations of nucleases and low pH. The particle penetrates the mucous membrane coating the cells of the gastrointestinal tract to reach the surface of the gastrointestinal tract. After reaching the surface, the particle releases the nucleic acid (e.g., in an unbound form or complexed with cationic lipids/polymer that uptake of the nucleic acids by the cell) or is taken up by the cell.

Expression of Therapeutic Protein

The expression of interferon β into the bloodstream as a result of administration of this particle can conveniently be used to treat disease (e.g., multiple sclerosis). The level and rate of gene expression can be adjusted as needed. Typically the nucleic acids are under control of the cytomegalovirus (CMV) promoter, but other promoters, i.e., tissue specific promoters may be used. For example, promoters that are effective in the epithelial cells of the gastrointestinal system can be used. The use of gut-specific promoters or any other plasmid DNA modifications may result in increased or tissue specific expression of interferon β in the gastrointestinal system. The details of plasmid design and manipulation are described in Example X above.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A particle, said particle comprising:
an active agent optionally in an aqueous interior;
an amphiphilic binding molecule; and
an encapsulation material, wherein said amphiphilic binding molecule comprises a first functionality and a second functionality, wherein said first functionality has an affinity for said active agent and said second functionality is soluble in the same solvent as said encapsulation material.

2. The particle of claim 1, wherein said active agent is nucleic acid.

3. The particle of claim 2, wherein said nucleic acid is selected from the group consisting of DNA, RNA, DNA/RNA hybrids, an antisense oligonucleotide, siRNA, a chimeric DNA-RNA polymer, a ribozyme, and a plasmid DNA.

4. The particle of claim 1, wherein said amphiphilic binding molecule is a cationic lipid.

5. The particle of claim 4, wherein said cationic lipid is selected from the group consisting of N,N-dioleyl-N,N-dimethylammonium chloride (“DODAC”), N-(2,3-dioleyloxy)propyl)-N,N,N-trimethylammonium chloride (“DOTMA”), N,N-distearyl-N,N-dimethylammonium bromide (“DDAB”), N-(2,3-dioleoyloxy)propyl)-N,N,N-trimethylammonium chloride (“DOTAP”), 1,2-dimyristoyl-sn-glycero-3-trimethylammonium-propane (“DMTAP”), 1,2-dipalmitoyl-sn-glycero-3-trimethylammonium-propane (“DPTAP”), and 1,2-distearoyl-sn-glycero-3-trimethylammonium-propane (“DSTAP”), 3-(N-(N′,N′-dimethylaminoethane)-carbamoyl)cholesterol (“DC-Chol”), N-(1,2-dimyristyloxyprop-3-yl)-N,N-dimethyl-N-hydroxyethyl ammonium bromide (“DMRIE”), 1,2-dilauroyl-P-O-ethylphosphatidylcholine (“E-DLPC”), 1,2-dimyristoyl-P-O-ethylphosphatidylcholine (“E-DMPC”), 1,2-dipalmitoyl-P-O-ethylphosphatidylcholine (“E-DPPC”), and mixtures thereof.

6. The particle of claim 1, wherein said encapsulation material is a hydrophobic polymer.

7. The particle of claim 6, wherein said hydrophobic polymer is a member selected from the group consisting of poly(lactid-co-glycolide), poly(lactic acid), poly(caprolactone), poly(glycolic-acid), poly(anhydrides), poly(orthoesters), poly (hydroxybutyric acid), poly (alkylcyanoacrylate), poly(lactides), poly(glycolides), poly(lactic acid-co-glycolic acid), polycarbonates, polyesteramides, poly(amino acids), polycyanoacrylates, poly(p-dioxanone), poly(alkylene oxalate), biodegradable polyurethanes, blends, and mixtures thereof.

8. The particle of claim 1, wherein said encapsulation material is a hydrophilic polymer.

9. The particle of claim 1, further comprising a stabilizing agent.

10. The particle of claim 9, wherein said stabilizing agent is selected from the group consisting of polyvinyl alcohol, methylcellulose, hydroxyethyl cellulose, hydroxypropylmethylcellulose, gelatin, a carbomer, and a poloxamer.

11. The particle of claim 2, wherein the ratio of said amphiphilic binding molecule to said nucleic acid is about 1:100 to about 20:1 w/w.

12. The particle of claim 11, wherein the ratio of said amphiphilic binding molecule to said nucleic acid is about 0.5:12 to about 10:1 w/w.

13. The particle of claim 12, wherein the ratio of said amphiphilic binding molecule to said nucleic acid is about 6:1 w/w.

14. The particle of claim 1, wherein said active agent is about 0.002% to about 50% w/w of said encapsulation material.

15. The particle of claim 14, wherein said active agent is about 0.01% to about 20% w/w of said encapsulation material.

16. The particle of claim 15, wherein said active agent is about 0.01% to about 10% w/w of said encapsulation material.

17. The particle of claim 1, wherein said particle has a diameter of about 0.1 μm to about 50 μm.

18. The particle of claim 17, wherein said particle has a diameter of about 0.5 μm to about 10 μm.

19. The particle of claim 1, further comprising an enteric coating.

20. The particle of claim 2, wherein said nucleic acid comprises a sequence encoding a therapeutic protein.

21. The particle of claim 20, wherein said therapeutic protein is selected from the group consisting of interferon α, interferon β, interferon γ, and insulin.

22. The particle of claim 20, wherein said therapeutic protein is interferon β.

23. The particle of claim 20, wherein said nucleic acid is operably linked to an expression control sequence.

24. The particle of claim 23, wherein said expression control sequence is tissue specific.

25. The particle of claim 24, wherein said tissue is intestinal epithelium.

26. The particle of claim 24, wherein said tissue is liver.

27. A process for preparing a particle, said process comprising:
admixing a first aqueous solution having an active agent with an organic solvent having an encapsulation material to form an emulsion;
admixing an amphiphilic binding molecule with said emulsion to form an amphiplex; and
admixing said amphiplex with a second aqueous solution having a stabilizing agent to form a particle, wherein said amphiphilic binding molecule comprises a first functionality and a second functionality, wherein said first functionality has an affinity for said active agent and said second functionality is soluble in the same solvent as said encapsulation material.

28. The process of claim 27, wherein said active agent is nucleic acid.

29. The process of claim 28, wherein said nucleic acid is selected from the group consisting of DNA, RNA, DNA/RNA hybrids, an antisense oligonucleotide, siRNA, a chimeric DNA-RNA polymer, a ribozyme, and a plasmid DNA.

30. The process of claim 27, wherein said encapsulation material is a hydrophobic polymer.

31. The process of claim 30, wherein said hydrophobic polymer is a member selected from the group consisting of poly(lactid-co-glycolide), poly(lactic acid), poly(caprolactone), poly(glycolic-acid), poly(anhydrides), poly(orthoesters), poly (hydroxybutyric acid), poly (alkylcyanoacrylate), poly(lactides), poly(glycolides), poly(lactic acid-co-glycolic acid), polycarbonates, polyesteramides, poly(amino acids), polycyanoacrylates, poly(p-dioxanone), poly(alkylene oxalate), biodegradable polyurethanes, blends, and mixtures thereof.

32. The process of claim 27, wherein said encapsulation material is a hydrophilic polymer.

33. The process of claim 27, wherein said amphiphilic binding molecule is a cationic lipid.

34. The process of claim 33, wherein said cationic lipid is selected from the group consisting of N,N-dioleyl-N,N-dimethylammonium chloride (“DODAC”), N-(2,3-dioleyloxy)propyl)-N,N,N-trimethylammonium chloride (“DOTMA”), N,N-distearyl-N,N-dimethylammonium bromide (“DDAB”), N-(2,3-dioleoyloxy)propyl)-N,N,N-trimethylammonium chloride (“DOTAP”), 1,2-dimyristoyl-sn-glycero-3-trimethylammonium-propane (“DMTAP”), 1,2-dipalmitoyl-sn-glycero-3-trimethylammonium-propane (“DPTAP”), and 1,2-distearoyl-sn-glycero-3-trimethylammonium-propane (“DSTAP”), 3-(N-(N′,N′-dimethylaminoethane)-carbamoyl)cholesterol (“DC-Chol”), N-(1,2-dimyristyloxyprop-3-yl)-N,N-dimethyl-N-hydroxyethyl ammonium bromide (“DMRIE”), 1,2-dilauroyl-P-O-ethylphosphatidylcholine (“E-DLPC”), 1,2-dimyristoyl-P-O-ethylphosphatidylcholine (“E-DMPC”), 1,2-dipalmitoyl-P-O-ethylphosphatidylcholine (“E-DPPC”), and mixtures thereof.

35. The process of claim 27, wherein increasing said amphiphilic binding molecule concentration decreases the diameter of said particle.

36. The process of claim 27, wherein increasing said amphiphilic binding molecule concentration increases encapsulation efficiency of said active agent.

37. The process of claim 27, wherein longer hydrophobic domains of said amphiphilic binding molecule decreases the diameter of said particle.

38. The process of claim 27, wherein longer hydrophobic domains of said amphiphilic binding molecule increases encapsulation efficiency of said active agent.

39. The process of claim 27, wherein said organic solution is selected from the group consisting of a hydrocarbon, an alkane, a halogenated alkane, acetone and petroleum ether.

40. The process of claim 27, wherein said stabilizing agent is selected from the group consisting of polyvinyl alcohol, methylcellulose, hydroxyethyl cellulose, hydroxypropylmethylcellulose, gelatin, a carbomer, and a poloxamer.

41. The process of claim 27, wherein said particle is about 0.01 μm to about 1000 μm in diameter.

42. The process of claim 27, further comprising lyophilizing said particle to form a delivery particle.

43. A particle prepared according to claim 42.

44. A delivery particle, said delivery particle comprising:
an inner core having an active agent;
an amphiphilic binding molecule; and
a polymeric outer layer, wherein said amphiphilic binding molecule is situated between said inner core and said outer layer.

45. The delivery particle of claim 44, wherein said inner core is a disperse phase.

46. The delivery particle of claim 44, wherein said inner core comprises a disperse phase, an active ingredient, or a mixture of an outer layer and an active ingredient.

47. The delivery particle of claim 44, wherein said polymeric outer layer is an organic phase.

48. A method for retaining a material in a first phase of a two phase system, said method comprising:
providing an amphiphilic binding molecule comprising a first functionality and a second functionality, wherein said first functionality has an affinity for said material in said first phase and said second functionality is soluble in a second phase; and
wherein said amphiphilic binding molecule is situated between said first phase and said second phase thereby retaining said material in said first phase.

49. The method of claim 48, wherein said first phase is a disperse phase.

50. The method of claim 48, wherein said second phase is immiscible in said first phase.

51. The method of claim 48, wherein said two phase system further comprises a third phase to generate a three phase system.

52. The method of claim 51, wherein said three phase system is an w₁/o/w₂ emulsion.

53. The method of claim 48, wherein said amphiphilic binding molecule is a cationic lipid.

54. The method of claim 48, wherein said material is an active agent.

55. The method of claim 54, wherein said active agent is nucleic acid.

56. A method for inducing an immune response in a subject, said method comprising administering a particle of claim 44 to the subject.

57. The method of claim 56, wherein said administration is oral.

58. The method of claim 56, wherein said active agent is nucleic acid.

59. The method of claim 58, wherein said nucleic acid is operably linked to an expression control sequence.

60. The method of claim 59, wherein said expression control sequence is tissue specific.

61. The method of claim 60, wherein said tissue is intestinal epithelium.

62. The method of claim 58, wherein said nucleic acid encodes a protein selected from the group consisting of a bacterial antigen, a viral antigen, a fungal antigen, and a parasitic antigen.

63. The method of claim 58, wherein said nucleic acid encodes a viral antigen.

64. The method of claim 58, wherein said nucleic acid encodes HIV gp120.
